(19) United States
(12) Patent Application Publication
(10) Pub. No.: US 2014/0057918 A1
(43) Pub. Date: Feb. 27, 2014

(54) Methods of Use for Monomethyl Fumarate and Prodrugs Thereof

(71) Applicant: XenoPort, Inc., Santa Clara, CA (US)

(72) Inventors: David J. Wustrow, Los Gatos, CA (US); Peter A. Virsik, Portola Valley, CA (US); Mark A. Gallop, Palo Alto, CA (US); Kenneth C. Cundy, Redwood City, CA (US); Dmitri V. Lissin, Santa Clara, CA (US); Revati Shreeniwas, Palo Alto, CA (US)

(73) Assignee: XenoPort, Inc., Santa Clara, CA (US)

(21) Appl. No.: 13/973,820

(22) Filed: Aug. 22, 2013

Related U.S. Application Data
(60) Provisional application No. 61/692,168, filed on Aug. 22, 2012.
(60) Provisional application No. 61/692,174, filed on Aug. 22, 2012.
(60) Provisional application No. 61/692,179, filed on Aug. 22, 2012.
(60) Provisional application No. 61/713,897, filed on Oct. 15, 2012.
(60) Provisional application No. 61/713,961, filed on Oct. 15, 2012.
(60) Provisional application No. 61/733,234, filed on Dec. 4, 2012.
(60) Provisional application No. 61/736,906, filed on Dec. 13, 2012.
(60) Provisional application No. 61/769,513, filed on Feb. 26, 2013.
(60) Provisional application No. 61/837,796, filed on Jun. 21, 2013.
(60) Provisional application No. 61/841,513, filed on Jul. 1, 2013.
(60) Provisional application No. 61/864,064, filed on Aug. 9, 2013.

(51) Int. Cl. C07C 69/60; C07D 265/30

(52) U.S. Cl. CPC C07C 69/60; C07D 265/30
USPC 514/239.2; 514/547

(57) ABSTRACT

Methods of therapeutic treatment using monomethyl fumarate and prodrugs of monomethyl fumarate are disclosed.

### CROSS REFERENCE TO RELATED APPLICATIONS

This application claims the benefit under 35 U.S.C. §119(e) of U.S. Provisional Application Ser. Nos. 61/736,906, filed Dec. 13, 2012; 61/864,064, filed Aug. 9, 2013; 61/692,179, filed Aug. 22, 2012; 61/692,168, filed Aug. 22, 2012; 61/713,897, filed Oct. 15, 2012; 61/733,234, filed Dec. 4, 2012; 61/769,513, filed Feb. 26, 2013; 61/841,513, filed Jul. 1, 2013; 61/692,174, filed Aug. 22, 2012; 61/713,961, filed Oct. 15, 2012; and 61/837,796, filed Jun. 21, 2013; the contents of each of which are incorporated herein by reference in their entirety.

### TECHNICAL FIELD

Disclosed herein are methods of using monomethyl fumarate and prodrugs thereof for treating various diseases.

### BACKGROUND

Fumaric acid esters (FAEs) are approved in Germany for the treatment of psoriasis, are being evaluated in the United States for the treatment of psoriasis and multiple sclerosis, and have been proposed for use in treating a wide range of immunological, autoimmune, and inflammatory diseases and conditions.

FAEs and other fumaric acid derivatives have been proposed for use in treating a wide-variety of diseases and conditions involving immunological, autoimmune, and/or inflammatory processes including psoriasis (Joshi and Strebel, WO 1999/49858; U.S. Pat. No. 6,277,882; Mrowietz and Asadullah, Trends Mol Med 2005, 111(1), 43-48; and Yazdi and Mrowietz, Clinics Dermatology 2008, 26, 522-526); asthma and chronic obstructive pulmonary diseases (Joshi et al., WO 2005/023241 and US 2007/0027076); cardiac insufficiency including left ventricular insufficiency, myocardial infarction and angina pectoris (Joshi et al., WO 2005/023241; Joshi et al., US 2007/0027076); mitochondrial and neurodegenerative diseases such as Parkinson's disease, Alzheimer's disease, Huntington's disease, retinopathia pigmentosa and mitochondrial encephalomyopathy (Joshi and Strebel, WO 2002/055063, US 2006/0205659, U.S. Pat. No. 6,509,376, U.S. Pat. No. 6,858,750, and U.S. Pat. No. 7,157,423); transplantation (Joshi and Strebel, WO 2002/055063, US 2006/0205659, U.S. Pat. No. 6,359,003, U.S. Pat. No. 6,509,376, and U.S. Pat. No. 7,157,423; and Lehmann et al., Arch Dermatol Res 2002, 294, 399-404); autoimmune diseases (Joshi and Strebel, WO 2002/055063, U.S. Pat. No. 6,509,376, U.S. Pat. No. 7,157,423, and US 2006/0205659) including multiple sclerosis (MS) (Joshi and Strebel, WO 1998/52549 and U.S. Pat. No. 6,436,992; Went and Lieberburg, US 2008/0089896; Schimrigk et al., Eur J Neurology 2006, 13, 604-610; and Schilling et al., Clin Experimental Immunology 2006, 145, 101-107); neurological disorders characterized by extensive demyelination and/or axonal loss including secondary progressive multiple sclerosis and Devic's disease (Gold, WO 2008/096271); ischemia and reperfusion injury (Joshi et al., US 2007/0027076); AGE-induced genome damage (Heidland, WO 2005/027899); inflammatory bowel diseases such as Crohn's disease and ulcerative colitis; arthritis; and others (Nilsson et al., WO 2006/037342 and Nilsson and Muller, WO 2007/042034).

The mechanism of action of fumaric acid esters is believed to be mediated by pathways associated with the immunological response. For example, FAEs: invoke a shift from a Th1 to Th2 immune response, favorably altering the cytokine profile; inhibit cytokine-induced expression of adhesion molecules such as VCAM-1, ICAM-1 and E-selectin, thereby reducing immune cell extravasation; and deplete lymphocytes through apoptotic mechanisms (Lehmann et al., J Investigative Dermatology 2007, 127, 835-845; Gesser et al., J Investigative Dermatology 2007, 127, 2129-2137; Vandermeeren et al., Biochm Biophys Res Commun 1997, 234, 19-23; and Treumer et al., J Invest Dermatol 2003, 121, 1383-1388).

Recent studies suggest that FAEs are inhibitors of NF-κB activation, a transcription factor that regulates the inducible expression of proinflammatory mediators (D'Acquisto et al., Molecular Interventions 2002, 2(1), 22-35). Accordingly, FAEs have been proposed for use in treating NF-κB mediated diseases (Joshi et al., WO 2002/055066; and Joshi and Strebel, WO 2002/055063, US 2006/0205659, U.S. Pat. No. 7,157,423 and U.S. Pat. No. 6,509,376). Inhibitors of NF-κB activation have also been shown to be useful in angiostatic therapy (Tabruyn and Griffioen, Angiogenesis 2008, 11, 101-106), inflammatory bowel disease (Atreya et al., J Intern Med 2008, 263(6), 591-6); and in animal models of diseases involving inflammation including neutrophilic alveolitis, asthma, hepatitis, inflammatory bowel disease, neurodegeneration, ischemia/reperfusion, septic shock, glomerulonephritis, and rheumatoid arthritis (D'Acquisto et al., Molecular Interventions 2002, 2(1), 22-35).

Studies also suggest that NF-κB inhibition by FAEs may be mediated by interaction with tumor necrosis factor (TNF) signaling. Dimethyl fumarate inhibits TNF-induced tissue factor mRNA and protein expression, TNF-induced DNA binding of NF-κB proteins, and the TNF-induced nuclear entry of activated NF-κB proteins, thereby inhibiting inflammatory gene activation (Loewe et al., J Immunology 2002, 168, 4781-4787). TNF signaling pathways are implicated in the pathogenesis of immune-mediated inflammatory diseases such as rheumatoid arthritis, Crohn's disease, psoriasis, psoriatic arthritis, juvenile idiopathic arthritis, and ankylosing spondylitis (Tracey et al., Pharmacology & Therapetuics 2008, 117, 244-279).

Fumaderm®, an enteric coated tablet containing a mixture of salts of monoethyl fumarate and dimethyl fumarate was approved in Germany in 1994 for the treatment of psoriasis. Dimethyl fumarate (DMF) is rapidly metabolized in vivo to monomethyl fumarate (MMF), and hence DMF is considered to be a prodrug of MMF.

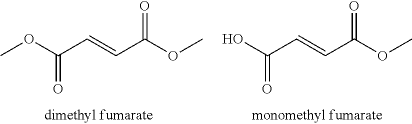

Fumaderm® is dosed three times per day with 1-2 grams/day administered for the treatment of psoriasis. Fumaderm® exhibits a high degree of interpatient variability with respect to drug absorption and food strongly reduces bioavailability. Absorption is thought to occur in the small intestine with peak levels achieved 5-6 hours after oral administration. Significant side effects occur in 70-90% of patients (Brewer and Rogers, Clin Expt'l Dermatology 2007, 32, 246-49; and Hoefnagel et al., Br J Dermatology 2003, 149, 363-369). Side effects of current FAE therapy include gastrointestinal upset including nausea, vomiting, and diarrhea; transient flushing of the skin. Also, DMF exhibits poor aqueous solubility.

Fumaric acid derivatives (Joshi and Strebel, WO 2002/055063, US 2006/0205659, and U.S. Pat. No. 7,157,423 (amide compounds and protein-fumarate conjugates); Joshi et al., WO 2002/055066 and Joshi and Strebel, U.S. Pat. No. 6,355,676 (mono and dialkyl esters); Joshi and Strebel, WO 2003/087174 (carbocyclic and oxacarbocylic compounds); Joshi et al., WO 2006/122652 (thiosuccinates); Joshi et al., US 2008/0233185 (dialkyl and diaryl esters) and Nilsson et al., US 2008/0004344 (salts)) have been developed in an effort to overcome the deficiencies of current FAE therapy. Controlled release pharmaceutical compositions comprising fumaric acid esters are disclosed by Nilsson and Müller, WO 2007/042034. Glycolamide ester prodrugs are described by Nielsen and Bundgaard, J Pharm Sci 1988, 77(4), 285-298.

Prodrugs of monomethyl fumarate and therapeutic uses thereof are disclosed in US Patent Publication US 2010/0048651 published Feb. 25, 2010.

Other prodrugs of monomethyl fumarate and therapeutic uses thereof are disclosed in US Patent Publication US 2013/0203753 published Aug. 8, 2013.

Fumaderm® (dimethyl fumarate and ethyl hydrogen fumarate) has previously been used to treat cutaneous lupus erythematosus. See for example Klein, A., et al. (2011), J Eur Acad Dermatol Venereol doi: 10.1111/j.1468-3083.2011.04303.x.

Dimethyl fumarate has previously been administered to animals infected with the herpes simplex virus and improved the animals' herpes stromal keratitis. See for example Heiligenhaus, A., et al. (2005), Clinical and Experimental Immunology 142(1): 180-187; Heiligenhaus, A., et al. (2004), Graefe's Archive for Clinical and Experimental Ophthalmology 242(10): 870-877.

Dimethyl fumarate and monomethyl fumarate have previously been suggested as a neuroprotectant in HIV patients. See for example Cross, S. A., et al. (2011), Journal of Immunology 187(10): 5015-5025.

Fumaderm® (dimethyl fumarate and ethyl hydrogen fumarate) and dimethyl fumarate alone have previously been used to treat lichen planus. See for example Guenther, C. H., et al. (2003), Annals of Pharmacotherapy 37(2): 234-236; and Klein, A., et al. (2012), Journal of the European Academy of Dermatology and Venereology 26(11): 1400-1406.

Based on in vitro testing on human retinal pigment epithelium cells, dimethyl fumarate has been suggested for use in treating macular degeneration. See for example Nelson, K. C., et al. (1999), Investigative Ophthalmology and Visual Science 40(9): 1927-1935; and Winkler, B. S., et al. (1999), Molecular vision 5: 32.

Fumaderm® (dimethyl fumarate and ethyl hydrogen fumarate) has previously been used to treat necrobiosis lipoidosis. See for example Eberle, F. C., et al. (2010), Acta Derm Venereol 90(1): 104-106; Gambichler, T., et al. (2003), Dermatology 207(4): 422-424; Kreuter, A., et al. (2005), British Journal of Dermatology 153(4): 802-807; and Wang, W. P., et al. (2007), Chinese Journal of Evidence-Based Medicine 7(11): 830-835.

Lukashev (WO 2010/126605) discloses administering fumaric acid esters such as monoalkyl and dialkyl fumarates for treating diseases including, among others, adrenal leukodystrophy, Alexander's disease, Alper's disease, Canavan disease, HIV-associated dementia, Krabbe's disease (globoid cell leukodystrophy), Pelizaeus-Merzbacher Disease, primary lateral sclerosis, progressive supranuclear palsy and Schilder's disease.

Joshi et al., U.S. Pat. No. 6,359,003, discloses methods for treating host-versus-graft reactions using fumaric acid derivatives, such as monoalkyl and dialkyl fumarates.

Joshi et al., US Patent Application Publication No. 2004/0054001, discloses using fumaric acid derivatives, such as monoalkyl and dialkyl fumarates, for treating diseases including, among others, pneumonia, inflammatory demyelinising polyneuropathy, hepatitis (acute hepatitis, chronic hepatitis, toxic hepatitis, alcohol-induced hepatitis, viral hepatitis, jaundice, liver insufficiency and cytomegaloviral hepatitis), AIDS, and cancers such as mamma carcinoma, colon carcinoma, melanoma, primary liver cell carcinoma, adenocarcinoma, kaposi's sarcoma, prostate carcinoma, leukaemia such as acute myeloid leukaemia, multiple myeloma (plasmocytoma), Burkitt lymphoma and Castleman tumour.

Nilsson et al. (WO 2010/079222) discloses using fumaric acid derivatives, such as monoalkyl and dialkyl fumarates, for treating diseases including, among others, systemic lupus erythematosus, chronic active (lupoid) hepatitis, optic neuritis, and organ transplantation (prevention of rejection).

Lukashev et al. (WO 2008/097596) discloses using fumaric acid derivatives, such as monoalkyl and dialkyl fumarates, for treating diseases including, among others, Zellweger syndrome.

Kahrs (US 2013/0172391) discloses the use of MMF and DMF for the treatment of diseases including, among others, chronic lymphocytic leukemia and macular degeneration.

Steinman et al. (WO 2013/022882) discloses the use of DMF in combination with an ACE inhibitor for the treatment of diseases including, among others, neuromyelitis optica.

### SUMMARY

Disclosed herein are methods of treating a disease comprising administering to a patient in need of such treatment a therapeutically effective amount of a compound selected from: (i) monomethyl fumarate, (ii) a prodrug of monomethyl fumarate, and/or (iii) a combination thereof, wherein the disease to be treated is chosen from balo concentric sclerosis, bronchiolitis obliterans organizing pneumonia, central nervous system vasculitis, Charcott-Marie-Tooth Disease, childhood ataxia with central nervous system hypomyelination, diabetic retinopathy, graft versus host disease, monomelic amyotrophy, neurodegeneration with brain iron accumulation, neurosarcoidosis, pareneoplastic syndromes, subacute necrotizing myelopathy, Susac syndrome and transverse myelitis.

Also disclosed herein are methods of treating a disease comprising administering to a patient in need of such treatment a therapeutically effective amount of a compound selected from: (i) monomethyl fumarate; (ii) a prodrug of monomethyl fumarate chosen from a compound of Formulae (I), (II) and (V); and/or (iii) a combination thereof, wherein the disease to be treated is chosen from cutaneous lupus erythematosus, lichen planus, macular degeneration, necrobiosis lipoidosis and neuromyelitis optica.

Also disclosed herein are methods of treating a disease comprising administering to a patient in need of such treatment a therapeutically effective amount of a monomethyl fumarate prodrug chosen from a compound of Formulae (I), (II) and (V), wherein the disease to be treated is chosen from adrenal leukodystrophy, Alexanders Disease, Alpers' Disease, Canavan disease, chronic inflammatory demyelinating polyneuropathy, chronic lymphocytic leukemia, globoid cell leukodystrophy, hepatitis C viral infection, herpes simplex viral infection, human immunodeficiency viral infection, optic neuritis, Pelizaeus-Merzbacher disease, primary lateral sclerosis, progressive supranuclear palsy, Schilder's Disease, a tumor and Zellweger syndrome.

In a first aspect, the compound being administered comprises monomethyl fumarate.

In a second aspect, the compound being administered comprises a prodrug of monomethyl fumarate.

In a third aspect, the prodrug of monomethyl fumarate comprises dimethyl fumarate.

In a fourth aspect, the prodrug of monomethyl fumarate comprises a compound of Formula (I):

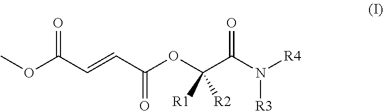

or a pharmaceutically acceptable salt thereof, wherein:

R¹ and R² are independently chosen from hydrogen, C₁₋₆ alkyl, and substituted C₁₋₆ alkyl; and

R³ and R⁴ are independently chosen from hydrogen, C₁₋₆ alkyl, substituted C₁₋₆ alkyl, C₁₋₆ heteroalkyl, substituted C₁₋₆ heteroalkyl, C₄₋₁₂ cycloalkylalkyl, substituted C₄₋₁₂ cycloalkylalkyl, C₇₋₁₂ arylalkyl, and substituted C₇₋₁₂ arylalkyl; or R³ and R⁴ together with the nitrogen to which they are bonded form a ring chosen from a C₅₋₁₀ heteroaryl, substituted C₅₋₁₀ heteroaryl, C₅₋₁₀ heterocycloalkyl, and substituted C₅₋₁₀ heterocycloalkyl;

wherein each substituent group is independently chosen from halogen, —OH, —CN, —CF₃, ═O, —NO₂, benzyl, —C(O)NR¹¹₂, —R¹¹, —OR¹¹, —C(O)R¹¹, —COOR¹¹, and —NR¹¹₂ wherein each R¹¹ is independently chosen from hydrogen and C₁₋₄ alkyl.

In a fifth aspect, the prodrug of monomethyl fumarate comprises a compound of Formula (II):

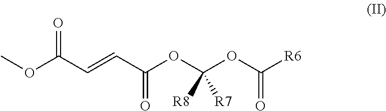

or a pharmaceutically acceptable salt thereof, wherein:

R⁶ is chosen from C₁₋₆ alkyl, substituted C₁₋₆ alkyl, C₁₋₆ heteroalkyl, substituted C₁₋₆ heteroalkyl, C₃₋₈ cycloalkyl, substituted C₃₋₈ cycloalkyl, C₆₋₈ aryl, substituted C₆₋₈ aryl, and —OR¹⁰ wherein R¹⁰ is chosen from C₁₋₆ alkyl, substituted C₁₋₆ alkyl, C₃₋₁₀ cycloalkyl, substituted C₃₋₁₀ cycloalkyl, C₆₋₁₀ aryl, and substituted C₆₋₁₀ aryl; and

R⁷ and R⁸ are independently chosen from hydrogen, C₁₋₆ alkyl, and substituted C₁₋₆ alkyl;

wherein each substituent group is independently chosen from halogen, —OH, —CN, —CF₃, ═O, —NO₂, benzyl, —C(O)NR¹¹₂, —R¹¹, —OR¹¹, —C(O)R¹¹, —COOR¹¹, and —NR¹¹₂ wherein each R¹¹ is independently chosen from hydrogen and C₁₋₄ alkyl.

In a sixth aspect, the prodrug of monomethyl fumarate comprises a compound of Formula (V):

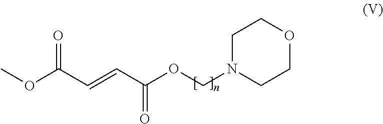

or a pharmaceutically acceptable salt thereof, wherein n is an integer from 2 to 6

### DETAILED DESCRIPTION

### Definitions

A dash (“—”) that is not between two letters or symbols is used to indicate a point of attachment for a moiety or substituent. For example, —CONH₂ is bonded through the carbon atom.

“Alkyl” refers to a saturated or unsaturated, branched, or straight-chain, monovalent hydrocarbon radical derived by the removal of one hydrogen atom from a single carbon atom of a parent alkane, alkene, or alkyne. Examples of alkyl groups include, but are not limited to, methyl; ethyls such as ethanyl, ethenyl, and ethynyl; propyls such as propan-1-yl, propan-2-yl, prop-1-en-1-yl, prop-1-en-2-yl, prop-2-en-1-yl (allyl), prop-1-yn-1-yl, prop-2-yn-1-yl, etc.; butyls such as butan-1-yl, butan-2-yl, 2-methyl-propan-1-yl, 2-methyl-propan-2-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, but-1-yn-1-yl, but-1-yn-3-yl, but-3-yn-1-yl, etc.; and the like.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds, and groups having combinations of single, double, and triple carbon-carbon bonds. Where a specific level of saturation is intended, the terms alkanyl, alkenyl, and alkynyl are used. In certain embodiments, an alkyl group can have from 1 to 20 carbon atoms (C₁₋₂₀) in certain embodiments, from 1 to 10 carbon atoms (C₁₋₁₀, in certain embodiments from 1 to 8 carbon atoms (C₁₋₈), in certain embodiments, from 1 to 6 carbon atoms (CO, in certain embodiments from 1 to 4 carbon atoms (C₁₋₄), and in certain embodiments, from 1 to 3 carbon atoms (C₁₋₃).

“Aryl” refers to a monovalent aromatic hydrocarbon radical derived by the removal of one hydrogen atom from a single carbon atom of a parent aromatic ring system. Aryl encompasses benzene; bicyclic ring systems wherein at least one ring is carbocyclic and aromatic, for example, naphthalene, indane, and tetralin; and tricyclic ring systems wherein at least one ring is carbocyclic and aromatic, for example, fluorene. Aryl encompasses multiple ring systems having at least one carbocyclic aromatic ring fused to at least one carbocyclic aromatic ring, cycloalkyl ring, or heterocycloalkyl ring. For example, aryl includes a phenyl ring fused to a 5- to 7-membered heterocycloalkyl ring containing one or more heteroatoms chosen from N, O, and S. For such fused, bicyclic ring systems wherein only one of the rings is a carbocyclic aromatic ring, the radical carbon atom may be at the carbocyclic aromatic ring or at the heterocycloalkyl ring. Examples of aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In certain embodiments, an aryl group can have from 6 to 20 carbon atoms (C₆₋₂₀), from 6 to 12 carbon atoms (C₆₋₁₂), from 6 to 10 carbon atoms (C₆₋₁₀), and in certain embodiments from 6 to 8 carbon atoms (C₆₋₈).

“Arylalkyl” refers to an acyclic alkyl radical in which one of the hydrogen atoms bonded to a carbon atom, typically a terminal or sp³ carbon atom, is replaced with an aryl group. Examples of arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl, or arylalkynyl is used. In certain embodiments, an arylalkyl group is C₇₋₃₀ arylalkyl, e.g., the alkanyl, alkenyl or alkynyl moiety of the arylalkyl group is C₁₋₁₀ and the aryl moiety is C₆₋₂₀, in certain embodiments, an arylalkyl group is C₆₋₁₈ arylalkyl, e.g., the alkanyl, alkenyl or alkynyl moiety of the arylalkyl group is C₁₋₈ and the aryl moiety is C₆₋₁₀. In certain embodiments, an arylalkyl group is C₇₋₁₂ arylalkyl.

“Compounds” of Formulae (I), (II) and (V) disclosed herein include any specific compounds within these formulae. Compounds may be identified either by their chemical structure and/or chemical name. Compounds are named using Chemistry 4-D Draw Pro, version 7.01c (ChemInnovation Software, Inc., San Diego, Calif.). When the chemical structure and chemical name conflict, the chemical structure is determinative of the identity of the compound. The compounds described herein may comprise one or more chiral centers and/or double bonds and therefore may exist as stereoisomers such as double-bond isomers (i.e., geometric isomers), enantiomers, or diastereomers. Accordingly, any chemical structures within the scope of the specification depicted, in whole or in part, with a relative configuration are deemed to encompass all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures may be resolved into their component enantiomers or stereoisomers using separation techniques or chiral synthesis techniques well known to the skilled artisan. Compounds selected from monomethyl fumarate, or a prodrug of monomethyl fumarate such as dimethyl fumarate or a compound of Formulae (I), (II) and (V), include, but are not limited to, optical isomers thereof, racemates thereof, and other mixtures thereof. In such embodiments, a single enantiomer or diastereomer, i.e., optically active form can be obtained by asymmetric synthesis or by resolution of the racemates. Resolution of the racemates may be accomplished, for example, by conventional methods such as crystallization in the presence of a resolving agent, or chromatography using, for example, chiral stationary phases. Notwithstanding the foregoing, in compounds selected from monomethyl fumarate, or a prodrug of monomethyl fumarate such as dimethyl fumarate or a compound of Formulae (I), (II) and (V), the configuration of the illustrated double bond is only in the E configuration (i.e. trans configuration).

Compounds selected from monomethyl fumarate, or a prodrug of monomethyl fumarate such as dimethyl fumarate or a compound of Formulae (I), (II) and (V), may also exist in several tautomeric forms including the enol form, the keto form, and mixtures thereof. Accordingly, the chemical structures depicted herein encompass all possible tautomeric forms of the illustrated compounds. Compounds selected from monomethyl fumarate, or a prodrug of monomethyl fumarate such as dimethyl fumarate or a compound of Formulae (I), (II) and (V), also include isotopically labeled compounds where one or more atoms have an atomic mass different from the atomic mass conventionally found in nature. Examples of isotopes that may be incorporated into the compounds disclosed herein include, but are not limited to, ²H, ³H, ¹¹C, ¹³C, ¹⁴C, ¹⁵N, ¹⁸O, ¹⁷O, etc. Compounds may exist in unsolvated forms as well as solvated forms, including hydrated forms and as N-oxides. In general, compounds as referred to herein may be free acid, hydrated, solvated, or N-oxides. Certain compounds may exist in multiple crystalline, co-crystalline, or amorphous forms. Compounds selected from monomethyl fumarate, or a prodrug of monomethyl fumarate such as dimethyl fumarate or a compound of Formulae (I), (II) and (V), include pharmaceutically acceptable salts thereof, or pharmaceutically acceptable solvates of the free acid form of any of the foregoing, as well as crystalline forms of any of the foregoing.

Compounds selected from monomethyl fumarate, or a prodrug of monomethyl fumarate such as dimethyl fumarate or a compound of any of Formulae (I), (II) and (V), also include solvates. A solvate refers to a molecular complex of a compound with one or more solvent molecules in a stoichiometric or non-stoichiometric amount. Such solvent molecules are those commonly used in the pharmaceutical art, which are known to be innocuous to a patient, e.g., water, ethanol, and the like. A molecular complex of a compound or moiety of a compound and a solvent can be stabilized by non-covalent intra-molecular forces such as, for example, electrostatic forces, van der Waals forces, or hydrogen bonds. The term “hydrate” refers to a solvate in which the one or more solvent molecules is water.

Further, when partial structures of the compounds are illustrated, an asterisk (*) indicates the point of attachment of the partial structure to the rest of the molecule.

“Cycloalkyl” refers to a saturated or partially unsaturated cyclic alkyl radical. Where a specific level of saturation is intended, the nomenclature cycloalkanyl or cycloalkenyl is used. Examples of cycloalkyl groups include, but are not limited to, groups derived from cyclopropane, cyclobutane, cyclopentane, cyclohexane, and the like. In certain embodiments, a cycloalkyl group is C₃₋₁₅ cycloalkyl, C₃₋₁₂ cycloalkyl, and in certain embodiments, C₃₋₈ cycloalkyl.

“Cycloalkylalkyl” refers to an acyclic alkyl radical in which one of the hydrogen atoms bonded to a carbon atom, typically a terminal or sp³ carbon atom, is replaced with a cycloalkyl group. Where specific alkyl moieties are intended, the nomenclature cycloalkylalkanyl, cycloalkylalkenyl, or cycloalkylalkynyl is used. In certain embodiments, a cycloalkylalkyl group is C₄₋₃₀ cycloalkylalkyl, e.g., the alkanyl, alkenyl, or alkynyl moiety of the cycloalkylalkyl group is C₁₋₁₀ and the cycloalkyl moiety is C₃₋₂₀, and in certain embodiments, a cycloalkylalkyl group is C₃₋₂₀ cycloalkylalkyl, e.g., the alkanyl, alkenyl, or alkynyl moiety of the cycloalkylalkyl group is C₁₋₈ and the cycloalkyl moiety is C₃₋₁₂. In certain embodiments, a cycloalkylalkyl group is C₄₋₁₂ cycloalkylalkyl.

“Dimethyl fumarate” refers to the dimethyl ester of fumaric acid. The compound has the formula H₃COOCCH═CHCOOCH₃, and has a molecular weight of 144.13 daltons. This compound is also known by the names Dimethyl (E)-butenedioate (IUPAC), trans-1,2-Ethylenedicarboxylic acid dimethyl ester and (E)-2-Butenedioic acid dimethyl ester. The compound is also referred to herein by the acronym DMF.

“Disease” refers to a disease, disorder, condition, or symptom of any of the foregoing.

“Drug” as defined under 21 U.S.C. §321(g)(1) means “(A) articles recognized in the official United States Pharmacopoeia, official Homeopathic Pharmacopoeia of the United States, or official National Formulary, or any supplement to any of them; and (B) articles intended for use in the diagnosis, cure, mitigation, treatment, or prevention of disease in man or other animals; and (C) articles (other than food) intended to affect the structure or any function of the body of man or other animals . . . .”

“Halogen” refers to a fluoro, chloro, bromo, or iodo group. In certain embodiments, halogen refers to a chloro group.

“Heteroalkyl” by itself or as part of another substituent refer to an alkyl group in which one or more of the carbon atoms (and certain associated hydrogen atoms) are independently replaced with the same or different heteroatomic groups. Examples of heteroatomic groups include, but are not limited to, —O—, —S—, —O—O—, —S—S—, —O—S—, —NR¹³, ═N—N═, —N═N—, —N═N—NR¹³—, —PR¹³—, —P(O)₂—, —POR¹³—, —O—P(O)₂—, —SO—, —SO₂—, —Sn(R¹³)₂—, and the like, where each R¹³ is independently chosen from hydrogen, C₁₋₆ alkyl, substituted C₁₋₆ alkyl, C₆₋₁₂ aryl, substituted C₆₋₁₂ aryl, C₇₋₁₈ arylalkyl, substituted C₇₋₁₈ arylalkyl, C₃₋₇ cycloalkyl, substituted C₃₋₇ cycloalkyl, C₃₋₇ heterocycloalkyl, substituted C₃₋₇ heterocycloalkyl, C₁₋₆ heteroalkyl, substituted C₁₋₆ heteroalkyl, C₆₋₁₂ heteroaryl, substituted C₆₋₁₂ heteroaryl, C₇₋₁₈ heteroarylalkyl, or substituted C₇₋₁₈ heteroarylalkyl. Reference to, for example, a C₁₋₆ heteroalkyl, means a C₁₋₆ alkyl group in which at least one of the carbon atoms (and certain associated hydrogen atoms) is replaced with a heteroatom. For example C₁₋₆ heteroalkyl includes groups having five carbon atoms and one heteroatom, groups having four carbon atoms and two heteroatoms, etc. In certain embodiments, each R¹³ is independently chosen from hydrogen and C₁₋₃ alkyl. In certain embodiments, a heteroatomic group is chosen from —O—, —S—, —NH—, —N(CH₃)—, and —SO₂—; and in certain embodiments, the heteroatomic group is —O—.

“Heteroaryl” refers to a monovalent heteroaromatic radical derived by the removal of one hydrogen atom from a single atom of a parent heteroaromatic ring system. Heteroaryl encompasses multiple ring systems having at least one heteroaromatic ring fused to at least one other ring, which can be aromatic or non-aromatic. For example, heteroaryl encompasses bicyclic rings in which one ring is heteroaromatic and the second ring is a heterocycloalkyl ring. For such fused, bicyclic heteroaryl ring systems wherein only one of the rings contains one or more heteroatoms, the radical carbon may be at the aromatic ring or at the heterocycloalkyl ring. In certain embodiments, when the total number of N, S, and O atoms in the heteroaryl group exceeds one, the heteroatoms are not adjacent to one another. In certain embodiments, the total number of heteroatoms in the heteroaryl group is not more than two.

Examples of heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, thiazolidine, oxazolidine, and the like. In certain embodiments, a heteroaryl group is from 4- to 20-membered heteroaryl (C₄₋₂₀), and in certain embodiments from 4- to 12-membered heteroaryl (C₄₋₁₀). In certain embodiments, heteroaryl groups are those derived from thiophene, pyrrole, benzothiophene, benzofuran, indole, pyridine, quinoline, imidazole, oxazole, or pyrazine. For example, in certain embodiments, C₅ heteroaryl can be furyl, thienyl, pyrrolyl, imidazolyl, pyrazolyl, isothiazolyl, isoxazolyl.

“Heterocycloalkyl” refers to a saturated or unsaturated cyclic alkyl radical in which one or more carbon atoms (and certain associated hydrogen atoms) are independently replaced with the same or different heteroatom; or to a parent aromatic ring system in which one or more carbon atoms (and certain associated hydrogen atoms) are independently replaced with the same or different heteroatom such that the ring system no longer contains at least one aromatic ring. Examples of heteroatoms to replace the carbon atom(s) include, but are not limited to, N, P, O, S, Si, etc. Examples of heterocycloalkyl groups include, but are not limited to, groups derived from epoxides, azirines, thiiranes, imidazolidine, morpholine, piperazine, piperidine, pyrazolidine, pyrrolidine, quinuclidine, and the like. In certain embodiments, a heterocycloalkyl group is C₅₋₁₀ heterocycloalkyl, C₅₋₈ heterocycloalkyl, and in certain embodiments, C₅₋₆ heterocycloalkyl.

“Leaving group” has the meaning conventionally associated with it in synthetic organic chemistry, i.e., an atom or a group capable of being displaced by a nucleophile and includes halogen such as chloro, bromo, fluoro, and iodo, acyloxy (alkoxycarbonyl) such as acetoxy and benzoyloxy, aryloxycarbonyl, mesyloxy, tosyloxy, trifluoromethanesulfonyloxy, aryloxy such as 2,4-dinitrophenoxy, methoxy, N,O-dimethylhydroxylamino, p-nitrophenolate, imidazolyl, and the like.

“Monomethyl fumarate” refers to the monomethyl ester of fumaric acid. The compound has the formula HOOCCH═CHCOOCH₃, and has a molecular weight of 130.10 daltons. The compound is also commonly referred to as 2(E)-Butenedioic acid 1-methyl ester, (2E)-4-Methoxy-4-oxobut-2-enoic acid; Fumaric acid hydrogen 1-methyl ester; (2E)-2-Butenedioic acid 1-methyl ester; (E)-2-Butenedioic acid monomethyl ester; Monomethyl trans-ethylene-1,2-dicarboxylate; and methyl hydrogen fumarate. The compound is also referred to herein and elsewhere by the acronyms MMF and/or MHF.

“Parent aromatic ring system” refers to an unsaturated cyclic or polycyclic ring system having a conjugated π (pi) electron system. Included within the definition of “parent aromatic ring system” are fused ring systems in which one or more of the rings are aromatic and one or more of the rings are saturated or unsaturated, such as, for example, fluorene, indane, indene, phenalene, etc. Examples of parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like.

“Parent heteroaromatic ring system” refers to an aromatic ring system in which one or more carbon atoms (and any associated hydrogen atoms) are independently replaced with the same or different heteroatom in such a way as to maintain the continuous π-electron system characteristic of aromatic systems and a number of out-of-plane π-electrons corresponding to the Hückel rule (4n+2). Examples of heteroatoms to replace the carbon atoms include, but are not limited to, N, P, O, S, and Si, etc. Specifically included within the definition of “parent heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Examples of parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, thiazolidine, oxazolidine, and the like.

“Patient” refers to a mammal, for example, a human.

“Pharmaceutically acceptable” refers to approved or approvable by a regulatory agency of the Federal or a state government or listed in the U.S. Pharmacopoeia or other generally recognized pharmacopoeia for use in animals, and more particularly in humans.

“Pharmaceutically acceptable salt” refers to a salt of a compound, which possesses the desired pharmacological activity of the parent compound. Such salts include acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; and salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine, and the like. In certain embodiments, a pharmaceutically acceptable salt is the hydrochloride salt. In certain embodiments, a pharmaceutically acceptable salt is the sodium salt.

“Pharmaceutically acceptable vehicle” refers to a pharmaceutically acceptable diluent, a pharmaceutically acceptable adjuvant, a pharmaceutically acceptable excipient, a pharmaceutically acceptable carrier, or a combination of any of the foregoing with which a compound provided by the present disclosure may be administered to a patient and which does not destroy the pharmacological activity thereof and which is non-toxic when administered in doses sufficient to provide a therapeutically effective amount of the compound.

“Pharmaceutical composition” refers to a compound selected from monomethyl fumarate, or a prodrug of monomethyl fumarate such as dimethyl fumarate or a compound of Formulae (I), (II) and (V), and at least one pharmaceutically acceptable vehicle, with which the compound is administered to a patient.

“Substituted” refers to a group in which one or more hydrogen atoms are independently replaced with the same or substituent group(s). In certain embodiments, each substituent group is independently chosen from halogen, —OH, —CN, —CF₃, ═O, —NO₂, benzyl, —C(O)NH₂, —R¹¹, —OR¹¹, —C(O)R¹¹, —COOR¹¹, and —NR¹¹₂ wherein each —R¹¹ is independently chosen from hydrogen and C₁₋₄ alkyl. In certain embodiments, each substituent group is independently chosen from halogen, —OH, —CN, —CF₃, —NO₂, benzyl, —R¹¹, —OR¹¹, and —NR¹¹₂ wherein each R¹¹ is independently chosen from hydrogen and C₁₋₄ alkyl. In certain embodiments, each substituent group is independently chosen from halogen, —OH, —CN, —CF₃, ═O, —NO₂, benzyl, —C(O)NR¹¹₂, —R¹¹, —OR¹¹, —C(O)R¹¹, —COOR¹¹, and —NR¹¹₂ wherein each R¹¹ is independently chosen from hydrogen and C₁₋₄ alkyl. In certain embodiments, each substituent group is independently chosen from —OH, C₁₋₄ alkyl, and —NH₂.

“Treating” or “treatment” of any disease refers to reversing, alleviating, arresting, or ameliorating a disease or at least one of the clinical symptoms of a disease, reducing the risk of acquiring at least one of the clinical symptoms of a disease, inhibiting the progress of a disease or at least one of the clinical symptoms of the disease or reducing the risk of developing at least one of the clinical symptoms of a disease. “Treating” or “treatment” also refers to inhibiting the disease, either physically, (e.g., stabilization of a discernible symptom), physiologically, (e.g., stabilization of a physical parameter), or both, and to inhibiting at least one physical parameter that may or may not be discernible to the patient. In certain embodiments, “treating” or “treatment” refers to protecting against or delaying the onset of at least one or more symptoms of a disease in a patient.

“Therapeutically effective amount” refers to the amount of a compound that, when administered to a subject for treating a disease, or at least one of the clinical symptoms of a disease, is sufficient to affect such treatment of the disease or symptom thereof. The “therapeutically effective amount” may vary depending, for example, on the compound, the disease and/or symptoms of the disease, severity of the disease and/or symptoms of the disease or disorder, the age, weight, and/or health of the patient to be treated, and the judgment of the prescribing physician. An appropriate amount in any given instance may be ascertained by those skilled in the art or capable of determination by routine experimentation.

“Therapeutically effective dose” refers to a dose that provides effective treatment of a disease or disorder in a patient. A therapeutically effective dose may vary from compound to compound, and from patient to patient, and may depend upon factors such as the condition of the patient and the route of delivery. A therapeutically effective dose may be determined in accordance with routine pharmacological procedures known to those skilled in the art.

Reference is now made in detail to certain embodiments of compounds, compositions, and methods. The disclosed embodiments are not intended to be limiting of the claims. To the contrary, the claims are intended to cover all alternatives, modifications, and equivalents.

### Compounds

Certain embodiments of the methods disclosed herein use an MMF prodrug of Formula (I):

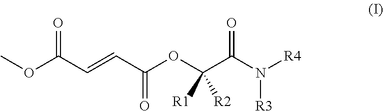

or a pharmaceutically acceptable salt thereof, wherein:

R¹ and R² are independently chosen from hydrogen, C₁₋₆ alkyl, and substituted C₁₋₆ alkyl; and

R³ and R⁴ are independently chosen from hydrogen, C₁₋₆ alkyl, substituted C₁₋₆ alkyl, C₁₋₆ heteroalkyl, substituted C₁₋₆ heteroalkyl, C₄₋₁₂ cycloalkylalkyl, substituted C₄₋₁₂ cycloalkylalkyl, C₇₋₁₂ arylalkyl, and substituted C₇₋₁₂ arylalkyl; or R³ and R⁴ together with the nitrogen to which they are bonded form a ring chosen from a C₅₋₁₀ heteroaryl, substituted C₅₋₁₀ heteroaryl, C₅₋₁₀ heterocycloalkyl, and substituted C₅₋₁₀ heterocycloalkyl;

wherein each substituent group is independently chosen from halogen, —OH, —CN, —CF₃, ═O, —NO₂, benzyl, —C(O)NR¹¹₂, —R¹¹, —OR¹¹, —C(O)R¹¹, —COOR¹¹, and —NR¹¹₂ wherein each R¹¹ is independently chosen from hydrogen and C₁₋₄ alkyl.

In certain embodiments of a method using a compound of Formula (I), each substituent group is independently chosen from halogen, —OH, —CN, —CF₃, —R¹¹, —OR¹¹, and —NR¹¹₂ wherein each R¹¹ is independently chosen from hydrogen and C₁₋₄ alkyl. In certain embodiments, each substituent group is independently chosen from —OH, and —COOH.

In certain embodiments of a method using a compound of Formula (I), each substituent group is independently chosen from ═O, C₁₋₄alkyl, and —COOR¹¹ wherein R¹¹ is chosen from hydrogen and C₁₋₄ alkyl.

In certain embodiments of a method using a compound of Formula (I), each of R¹ and R² is hydrogen.

In certain embodiments of a method using a compound of Formula (I), one of R¹ and R² is hydrogen and the other of R¹ and R² is C₁₋₄alkyl.

In certain embodiments of a method using a compound of Formula (I), one of R¹ and R² is hydrogen and the other of R¹ and R² is chosen from methyl, ethyl, n-propyl, isopropyl, n-butyl, isobutyl, sec-butyl, and tert-butyl.

In certain embodiments of a method using a compound of Formula (I), one of R¹ and R² is hydrogen and the other of R¹ and R² is methyl.

In certain embodiments of a method using a compound of Formula (I), R³ and R⁴ are independently chosen from hydrogen and C₁₋₆alkyl.

In certain embodiments of a method using a compound of Formula (I), R³ and R⁴ are independently chosen from hydrogen and C₁₋₄ alkyl.

In certain embodiments of a method using a compound of Formula (I), R³ and R⁴ are independently chosen from hydrogen, methyl, and ethyl.

In certain embodiments of a method using a compound of Formula (I), each of R³ and R⁴ is hydrogen; in certain embodiments, each of R³ and R⁴ is methyl; and in certain embodiments, each of R³ and R⁴ is ethyl.

In certain embodiments of a method using a compound of Formula (I), R³ is hydrogen; and R⁴ is chosen from C₁₋₄ alkyl, substituted C₁₋₄ alkyl wherein the substituent group is chosen from ═O, —OR¹¹, —COOR¹¹, and —NR¹¹₂, wherein each R¹¹ is independently chosen form hydrogen and C₁₋₄ alkyl.

In certain embodiments of a method using a compound of Formula (I), R³ is hydrogen; and R⁴ is chosen from C₁₋₄ alkyl, benzyl, 2-methoxyethyl, carboxymethyl, carboxypropyl, 1,2,4-thiadoxolyl, methoxy, 2-methoxycarbonyl, 2-oxo(1,3-oxazolidinyl), 2-(methylethoxy)ethyl, 2-ethoxyethyl, (tert-butyloxycarbonyl)methyl, (ethoxycarbonyl)methyl, carboxymethyl, (methylethyl)oxycarbonylmethyl, and ethoxycarbonylmethyl.

In certain embodiments of a method using a compound of Formula (I), R³ and R⁴ together with the nitrogen to which they are bonded form a ring chosen from a C₅₋₆ heterocycloalkyl, substituted C₅₋₆heterocycloalkyl, C₅₋₆heteroaryl, and substituted C₅₋₆ heteroaryl ring. In certain embodiments of a compound of Formula (I), R³ and R⁴ together with the nitrogen to which they are bonded form a ring chosen from a C₅ heterocycloalkyl, substituted C₅ heterocycloalkyl, C₅ heteroaryl, and substituted C₅ heteroaryl ring. In certain embodiments of a compound of Formula (I), R³ and R⁴ together with the nitrogen to which they are bonded form a ring chosen from a C₆ heterocycloalkyl, substituted C₆ heterocycloalkyl, C₆ heteroaryl, and substituted C₆ heteroaryl ring. In certain embodiments of a compound of Formula (I), R³ and R⁴ together with the nitrogen to which they are bonded form a ring chosen from piperazine, 1,3-oxazolidinyl, pyrrolidine, and morpholine ring.

In certain embodiments of a method using a compound of Formula (I), R³ and R⁴ together with the nitrogen to which they are bonded form a C₅₋₁₀ heterocycloalkyl ring.

In certain embodiments of a method using a compound of Formula (I), one of R¹ and R² is hydrogen and the other of R¹ and R² is C₁₋₆ alkyl; R³ is hydrogen; and R⁴ is chosen from hydrogen, C₁₋₆ alkyl, and benzyl.

In certain embodiments of a method using a compound of Formula (I), one of R¹ and R² is hydrogen and the other of R¹ and R² is C₁₋₆ alkyl; R³ is hydrogen; and R⁴ is chosen from hydrogen, C₁₋₆ alkyl, and benzyl.

In certain embodiments of a method using a compound of Formula (I), one of R¹ and R² is hydrogen and the other of R¹ and R² is chosen from hydrogen and C₁₋₆ alkyl; and each of R³ and R⁴ is C₁₋₆ alkyl.

In certain embodiments of a method using a compound of Formula (I), one of R¹ and R² is hydrogen and the other of R¹ and R² is chosen from hydrogen and C₁₋₆ alkyl; and each of R³ and R⁴ is C₁₋₆ alkyl. In certain embodiments of a compound of Formula (I), each of R¹ and R² is hydrogen; and each of R³ and R⁴ is C₁₋₆ alkyl.

In certain embodiments of a method using a compound of Formula (I), one of R¹ and R² is hydrogen and the other of R¹ and R² is chosen from hydrogen and C₁₋₄ alkyl; R³ is hydrogen; R⁴ is chosen from C₁₋₄ alkyl and substituted C₁₋₄ alkyl, wherein the substituent group is chosen from ═O, —OR¹¹, —COOR¹¹, and —NR¹¹₂, wherein each R¹¹ is independently chosen form hydrogen and C₁₋₄ alkyl. In certain embodiments of a method using a compound of Formula (I), one of R¹ and R² is hydrogen and the other of R¹ and R² is methyl; R³ is hydrogen; R⁴ is chosen from C₁₋₄ alkyl and substituted C₁₋₄ alkyl, wherein the substituent group is chosen from ═O, —OR¹¹, —COOR¹¹, and —NR¹¹₂, wherein each R¹¹ is independently chosen form hydrogen and C₁₋₄ alkyl. In certain embodiments of a method using a compound of Formula (I), each of R¹ and R² is hydrogen; R³ is hydrogen; R⁴ is chosen from C₁₋₄ alkyl and substituted C₁₋₄ alkyl, wherein the substituent group is chosen from ═O, —OR¹¹, —COOR¹¹, and —NR¹¹₂, wherein each R¹¹ is independently chosen form hydrogen and C₁₋₄ alkyl.

In certain embodiments of a method using a compound of Formula (I), R³ and R⁴ together with the nitrogen to which they are bonded form a C₅₋₁₀ heterocycloalkyl ring.

In certain embodiments of a method using a compound of Formula (I), one of R¹ and R² is hydrogen and the other of R¹ and R² is chosen from hydrogen and C₁₋₆ alkyl; and R³ and R⁴ together with the nitrogen to which they are bonded form a ring chosen from a C₅₋₆heterocycloalkyl, substituted C₅₋₆heterocycloalkyl, C₅₋₆heteroaryl, and substituted C₅₋₆heteroaryl ring. In certain embodiments of a method using a compound of Formula (I), one of R¹ and R² is hydrogen and the other of R¹ and R² is methyl; and R³ and R⁴ together with the nitrogen to which they are bonded form a ring chosen from a C₅₋₆heterocycloalkyl, substituted C₅₋₆heterocycloalkyl, C₅₋₆heteroaryl, and substituted C₅₋₆heteroaryl ring. In certain embodiments of a method using a compound of Formula (I), each of R¹ and R² is hydrogen; and R³ and R⁴ together with the nitrogen to which they are bonded form a ring chosen from a C₅₋₆heterocycloalkyl, substituted C₅₋₆heterocycloalkyl, C₅₋₆heteroaryl, and substituted C₅₋₆heteroaryl ring.

In certain embodiments of a method using a compound of Formula (I), one of R¹ and R² is hydrogen and the other of R¹ and R² is chosen from hydrogen and C₁₋₆ alkyl; and R³ and R⁴ together with the nitrogen to which they are bonded form a ring chosen from morpholine, piperazine, and N-substituted piperazine.

In certain embodiments of a method using a compound of Formula (I), one of R¹ and R² is hydrogen and the other of R¹ and R² is chosen from hydrogen and C₁₋₆ alkyl; and R³ and R⁴ together with the nitrogen to which they are bonded form a ring chosen from morpholine, piperazine, and N-substituted piperazine.

In certain embodiments of a method using a compound of Formula (I), R¹ is hydrogen, and in certain embodiments, R² is hydrogen.

In certain embodiments of a method using a compound of Formula (I), the compound is chosen from:
(N,N-diethylcarbamoyl)methyl methyl(2E)but-2-ene-1,4-dioate;
methyl [N-benzylcarbamoyl]methyl (2E)but-2-ene-1,4-dioate;
methyl 2-morpholin-4-yl-2-oxoethyl (2E)but-2-ene-1,4-dioate;
(N-butylcarbamoyl)methyl methyl(2E)but-2-ene-1,4-dioate;
[N-(2-methoxyethyl)carbamoyl]methyl methyl(2E)but-2-ene-1,4-dioate;
2-{2-[(2E)-3-(methoxycarbonyl)prop-2-enoyloxy]acetylamino}acetic acid;
4-{2-[(2E)-3-(methoxycarbonyl)prop-2-enoyloxy]acetylamino}butanoic acid;
methyl(N-(1,3,4-thiadiazol-2yl)carbamoyl)methyl(2E)but-2ene-1,4-dioate;
(N,N-dimethylcarbamoyl)methyl methyl(2E)but-2-ene-1,4-dioate;
(N-methoxy-N-methylcarbamoyl)methyl methyl(2E)but-2-ene-1,4-dioate; bis-(2-methoxyethylamino)carbamoyl]methyl methyl(2E)but-2-ene-1,4-dioate;
[N-(methoxycarbonyl)carbamoyl]methyl methyl(2E)but-2ene-1,4-dioate;
methyl 2-oxo-2-piperazinylethyl (2E)but-2-ene-1,4-dioate;
methyl 2-oxo-2-(2-oxo(1,3-oxazolidin-3yl)ethyl (2E)but-2ene-1,4-dioate;
{N-[2-(dimethylamino)ethyl]carbamoyl}methyl methyl(2E)but-2ene-1,4-dioate;
methyl 2-(4-methylpiperazinyl)-2-oxoethyl (2E)but-2-ene-1.4-dioate;
methyl {N-[(propylamino)carbonyl]carbamoyl}methyl (2E)but-2ene-1,4-dioate;
2-(4-acetylpiperazinyl)-2-oxoethyl methyl (2E)but-2ene-1,4-dioate;
{N,N-bis[2-(methylethoxy)ethyl]carbamoyl}methyl methyl(2E)but-2-ene-1,4-dioate;
methyl 2-(4-benzylpiperazinyl)-2-oxoethyl (2E)but-2-ene-1.4-dioate;
[N,N-bis(2-ethoxyethyl)carbamoyl]methyl methyl(2E)but-2-ene-1,4-dioate;
2-{(2S)-2-[(tert-butyl)oxycarbonyl]pyrrolidinyl}-2-oxoethyl methyl (2E)but-2ene-1,4-dioate;
1-{2-{(2E)-3-(methoxycarbonyl)prop-2-enoyloxy]acetyl}(2S)pyrrolidine-2-carboxylic acid;
(N-{[tert-butyl)oxycarbonyl]methyl}-N-methylcarbamoyl)methyl methyl(2E)but-2ene1,4-dioate;
{N-(ethoxycarbonyl)methyl]-N-methylcarbamoyl}methyl methyl(2E)but-2-ene-1,4-dioate;
methyl 1-methyl-2-morpholin-4-yl-2-oxoethyl (2E)but-2-ene-1,4-dioate;
[N,N-bis(2-methoxyethyl)carbamoyl]ethyl methyl (2E)but-2-ene-1,4-dioate;
(N,N-dimethylcarbamoyl)ethyl methyl (2E)but-2-ene-1,4-dioate;
(N-{[(tert-butyl)oxycarbonyl]methyl}carbamoyl)methyl methyl(2E)but-2-ene-1,4-dioate;
methyl (N-methyl-N-{[(methylethyl)oxycarbonyl]methyl}carbamoyl)methyl (2E)but-2-ene-1,4-dioate;
{N-[(ethoxycarbonyl)methyl]-N-benzylcarbamoyl}methyl methyl(2E)but-2-ene-1,4-dioate;
{N-[(ethoxycarbonyl)methyl]-N-benzylcarbamoyl}ethyl methyl (2E)but-2-ene-1,4-dioate;
{N-[(ethoxycarbonyl)methyl]-N-methylcarbamoyl}ethyl methyl (2E)but-2-ene-1,4-dioate;
(1S)-1-methyl-2-morpholin-4-yl-2-oxoethyl methyl (2E)but-2-ene-1,4-dioate;
(1S)-1-[N,N-bis(2-methoxyethyl)carbamoyl]ethyl methyl (2E)but-2-ene-1,4-dioate;
(1R)-1-(N,N-diethylcarbamoyl)ethyl methyl (2E)but-2-ene-1,4-dioate; and

a pharmaceutically acceptable salt of any of the foregoing.

In certain embodiments of a method using a compound of Formula (I), the compound is chosen from:
(N,N-diethylcarbamoyl)methyl methyl(2E)but-2-ene-1,4-dioate;
methyl [N-benzylcarbamoyl]methyl (2E)but-2-ene-1,4-dioate;
methyl 2-morpholin-4-yl-2-oxoethyl (2E)but-2-ene-1,4-dioate;
(N-butylcarbamoyl)methyl methyl(2E)but-2-ene-1,4-dioate;
[N-(2-methoxyethyl)carbamoyl]methyl methyl(2E)but-2-ene-1,4-dioate;
methyl(N-(1,3,4-thiadiazol-2yl)carbamoyl)methyl(2E)but-2ene-1,4-dioate;
(N,N-dimethylcarbamoyl)methyl methyl(2E)but-2-ene-1,4-dioate;
(N-methoxy-N-methylcarbamoyl)methyl methyl(2E)but-2-ene-1,4-dioate;
bis-(2-methoxyethylamino)carbamoyl]methyl methyl(2E)but-2-ene-1,4-dioate;
[N-(methoxycarbonyl)carbamoyl]methyl methyl(2E)but-2ene-1,4-dioate;
methyl 2-oxo-2-piperazinylethyl (2E)but-2-ene-1,4-dioate;
methyl 2-oxo-2-(2-oxo(1,3-oxazolidin-3yl)ethyl (2E)but-2ene-1,4-dioate;
{N-[2-(dimethylamino)ethyl]carbamoyl}methyl methyl(2E)but-2ene-1,4-dioate;
(N-[(methoxycarbonyl)ethyl]carbamoyl)methyl methyl(2E)but-2-ene-1,4-dioate;

and

a pharmaceutically acceptable salt of any of the foregoing.

Certain embodiments of the methods disclosed herein use an MMF prodrug of Formula (I), wherein R³ and R⁴ are independently chosen from hydrogen, C₁₋₆ alkyl, substituted C₁₋₆ alkyl, C₆₋₁₀ aryl, substituted C₆₋₁₀ aryl, C₄₋₁₂ cycloalkylalkyl, substituted C₄₋₁₂ cycloalkylalkyl, C₇₋₁₂ arylalkyl, substituted C₇₋₁₂ arylalkyl, C₁₋₆ heteroalkyl, substituted C₁₋₆ heteroalkyl, C₆₋₁₀ heteroaryl, substituted C₆₋₁₀ heteroaryl, C₄₋₁₂ heterocycloalkylalkyl, substituted C₄₋₁₂ heterocycloalkylalkyl, C₇₋₁₂ heteroarylalkyl, substituted C₇₋₁₂ heteroarylalkyl; or R³ and R⁴ together with the nitrogen to which they are bonded form a ring chosen from a C₅₋₁₀ heteroaryl, substituted C₅₋₁₀ heteroaryl, C₅₋₁₀ heterocycloalkyl, and substituted C₅₋₁₀ heterocycloalkyl.

Certain embodiments of the methods disclosed herein use an MMF prodrug of Formula (II):

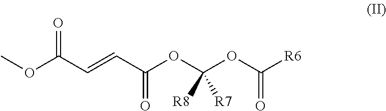

or a pharmaceutically acceptable salt thereof, wherein:

R⁶ is chosen from C₁₋₆alkyl, substituted C₁₋₆alkyl, C₁₋₆heteroalkyl, substituted C₁₋₆ heteroalkyl, C₃₋₈cycloalkyl, substituted C₃₋₈cycloalkyl, C₆₋₈aryl, substituted C₆₋₈aryl, and —OR¹⁰ wherein R¹⁰ is chosen from C₁₋₆alkyl, substituted C₁₋₆alkyl, C₃₋₁₀ cycloalkyl, substituted C₃₋₁₀ cycloalkyl, C₆₋₁₀ aryl, and substituted C₆₋₁₀ aryl; and

R⁷ and R⁸ are independently chosen from hydrogen, C₁₋₆alkyl, and substituted C₁₋₆ alkyl;

wherein each substituent group is independently chosen from halogen, —OH, —CN, —CF₃, ═O, —NO₂, benzyl, —C(O)NR¹¹₂, —R¹¹, —OR¹¹, —C(O)R¹¹, —COOR¹¹, and —NR¹¹₂ wherein each R¹¹ is independently chosen from hydrogen and C₁₋₄ alkyl.

In certain embodiments of a method using a compound of Formula (II), each substituent group is independently chosen from halogen, —OH, —CN, —CF₃, —R¹¹, —OR¹¹, and —NR¹¹₂ wherein each R¹¹ is independently chosen from hydrogen and C₁₋₄ alkyl.

In certain embodiments of a method using a compound of Formula (I), each substituent group is independently chosen from ═O, C₁₋₄alkyl, and —COOR¹¹ wherein R¹¹ is chosen from hydrogen and C₁₋₄alkyl.

In certain embodiments of a method using a compound of Formula (II), one of R⁷ and R⁸ is hydrogen and the other of R⁷ and R⁸ is C₁₋₆alkyl. In certain embodiments of a compound of Formula (II), one of R⁷ and R⁸ is hydrogen and the other of R⁷ and R⁸ is C₁₋₄alkyl.

In certain embodiments of a method using a compound of Formula (II), one of R⁷ and R⁸ is hydrogen and the other of R⁷ and R⁸ is chosen from methyl, ethyl, n-propyl, and isopropyl. In certain embodiments of a method using a compound of Formula (II), each of R⁷ and R⁸ is hydrogen.

In certain embodiments of a method using a compound of Formula (II), R⁶ is C₁₋₆alkyl; and one of R⁷ and R⁸ is hydrogen and the other of R⁷ and R⁸ is C₁₋₆alkyl.

In certain embodiments of a method using a compound of Formula (II), R⁶ is —OR¹⁰.

In certain embodiments of a method using a compound of Formula (II), R¹⁰ is chosen from C₁₋₄ alkyl, cyclohexyl, and phenyl.

In certain embodiments of a method using a compound of Formula (II), R⁶ is chosen from methyl, ethyl, n-propyl, and isopropyl; one of R⁷ and R⁸ is hydrogen and the other of R⁷ and R⁸ is chosen from methyl, ethyl, n-propyl, and isopropyl.

In certain embodiments of a method using a compound of Formula (II), R⁶ is substituted C₁₋₂ alkyl, wherein each of the one or more substituent groups are chosen from —COOH, —NHC(O)CH₂NH₂, and —NH₂.

In certain embodiments of a method using a compound of Formula (II), R⁶ is chosen from ethoxy, methylethoxy, isopropyl, phenyl, cyclohexyl, cyclohexyloxy, —CH(NH₂)CH₂COOH, —CH₂CH(NH₂)COOH, —CH(NHC(O)CH₂NH₂)—CH₂COOH, and —CH₂CH(NHC(O)CH₂NH₂)—COOH.

In certain embodiments of a method using a compound of Formula (II), one of R⁷ and R⁸ is hydrogen and the other of R⁷ and R⁸ is chosen from hydrogen, methyl, ethyl, n-propyl, and isopropyl; and R⁶ is chosen from C₁₋₃alkyl and substituted C₁₋₃ alkyl, wherein each of the one or more substituent groups are chosen from —COOH, —NHC(O)CH₂NH₂, and —NH₂, —OR¹⁰ wherein R¹⁰ is chosen from C₁₋₃alkyl and cyclohexyl, phenyl, and cyclohexyl.

In certain embodiments of a method using a compound of Formula (II), the compound is chosen from:
ethoxycarbonyloxyethyl methyl (2E)but-2-ene-1,4-dioate;
methyl (methylethoxycarbonyloxy)ethyl (2E)but-2-ene-1,4-dioate;
(cyclohexyloxycarbonyloxy)ethyl methyl (2E)but-2-ene-1,4-dioate; and

a pharmaceutically acceptable salt of any of the foregoing.

In certain embodiments of a method using a compound of Formula (II), the compound is chosen from:
methyl (2-methylpropanoyloxy)ethyl (2E)but-2-ene-1,4-dioate;
methyl phenylcarbonyloxyethyl (2E)but-2-ene-1,4-dioate;
cyclohexylcarbonyloxybutyl methyl (2E)but-2-ene-1,4-dioate;
[(2E)-3-(methoxycarbonyl)prop-2-enoyloxy]ethyl methyl (2E)but-2-ene-1,4-dioate;
methyl 2-methyl-1-phenylcarbonyloxypropyl (2E)but-2-ene-1,4-dioate; and

a pharmaceutically acceptable salt of any of the foregoing.

In certain embodiments of a method using a compound of Formula (II), the compound is chosen from:
(cyclohexyloxycarbonyloxy)ethyl methyl (2E)but-2-ene-1,4-dioate;
3-({[(2E)-3-(methoxycarbonyl)prop-2-enoyloxy]methyl}oxycarbonyl)(3S)-3-aminopropanoic acid;
3-({[(2E)-3-(methoxycarbonyl)prop-2-enoyloxy]methyl}oxycarbonyl)(2S)-2-aminopropanoic acid;
3-({[(2E)-3-(methoxycarbonyl)prop-2-enoyloxy]methyl}oxycarbonyl)(3S)-3-(2-aminoacetylamino)propanoic acid;
3-({[(2E)-3-(methoxycarbonyl)prop-2-enoyloxy]methyl}oxycarbonyl)(2S)-2-aminopropanoic acid;
3-{[(2E)-3-(methoxycarbonyl)prop-2enoyloxy]ethoxycarbonyloxy}(2S)-2-aminopropanoic acid; and

a pharmaceutically acceptable salt (e.g., a trifluoroacetate or a hydrochloride salt) of any of the foregoing.

Certain embodiments of the methods disclosed herein use an MMF prodrug of Formula (V):

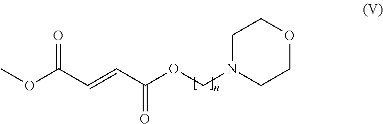

or a pharmaceutically acceptable salt thereof, wherein n is an integer from 2 to 6.

In certain embodiments of a method using a compound of Formula (V), n is 2, n is 3, n is 4, n is 5, and in certain embodiments, n is 6.

In certain embodiments of a method using a compound of Formula (V), the compound is a pharmaceutically acceptable salt.

In certain embodiments of a method using a compound of Formula (V), the compound is the hydrochloride salt.

In certain embodiments of a method using a compound of Formula (V), the compound is chosen from:
methyl (2-morpholinoethyl)fumarate;
methyl (3-morpholinopropyl)fumarate;
methyl (4-morpholinobutyl)fumarate;
methyl (5-morpholinopentyl)fumarate;
methyl (6-morpholinohexyl)fumarate;

or a pharmaceutically acceptable salt of any of the foregoing.

In certain embodiments, any one or more of the compounds of Formula (V) is a hydrochloride salt.

### Synthesis

Monomethyl fumarate prodrug compounds disclosed herein may be obtained via the synthetic methods illustrated in Schemes 1 through 8. General synthetic methods useful in the synthesis of compounds described herein are available in the art. Starting materials useful for preparing compounds and intermediates thereof and/or practicing methods described herein are commercially available or can be prepared by well-known synthetic methods. The methods presented in the schemes provided by the present disclosure are illustrative rather than comprehensive. It will be apparent to those skilled in the art that many modifications, both to materials and methods, may be practiced without departing from the scope of the disclosure.

Certain of the unsubstituted, 1-mono-substituted or 1,1-bis-substituted halo acetamides useful for preparing compounds of Formula (I) are available from commercial sources. Non-commercially available unsubstituted, 1-mono-substituted or 1,1-bis-substituted halo acetamides useful for preparing compounds of Formula (I) and intermediates thereof can be prepared by well-known synthetic methods such as those described in Schemes 1 and 2.

Functionalized 1-halo acetamides useful for the preparation of MMF acetamide prodrugs of Formula (I) can be prepared according to Scheme 1:

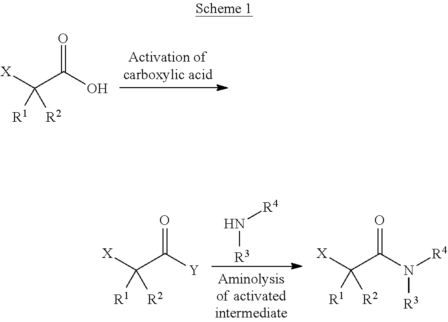

wherein X and Y are leaving groups such as halogen, and R¹, R², R³, and R⁴ are as defined herein. In certain embodiments of Scheme 1, X is chloro and Y is chloro or an O-acylisourea.

Chemical activation of the carboxylic acid to the corresponding carboxylic acid chloride as shown in Scheme 1 can be achieved by reaction with chlorination agents such as thionyl chloride (SOCl₂), oxalyl chloride (C₂O₂Cl₂), or phosphorous pentachloride (PCl₅), optionally in the presence of a suitable catalyst such as N,N-dimethylformamide (DMF), and either in substance (absence of solvent) or in an inert organic solvent such as dichloromethane (DCM) at an appropriate temperature such as from about 0° C. to about 70° C. Chemical activation of the carboxylic acid can be performed in situ and without isolating the activated substrate prior to the following aminolysis step. Optionally, the activated carboxylic acid can be isolated and/or purified using methods well known in the art, i.e. fractional distillation.

Alternatively, carbodiimide dehydration agents such as N,N′-diisopropylcarbodiimide (DIC), N,N′-dicyclohexylcarbodiimide (DCC), or 1-ethyl-3-(3-dimethylaminopropyl) carbodiimide (EDAC, EDC), optionally in the presence of a catalytic or stoichiometric amount of a suitable additive such as 4-(N,N-dimethylaminopyridine (DMAP) (Steglich esterification conditions), 1-hydroxybenzotriazole (HOBt), 1-hydroxy-7-aza-benzotriazole (HOAt), or N-hydroxysuccinimide (NHS); uronium or phosphonium salts with non-nucleophilic anions such as N-[(1H-benzotriazol-1-yl)(dimethylamino)methylene]-N-methylmetanaminium hexafluorophosphate (HBTU), N-[(dimethylamino)-1H-1,2,3-triazolo[4,5-b]pyridin-1-ylmethylene]-N-methylmetanaminium hexafluorophosphate N-oxide (HATU), N-[(1H-benzotriazol-1-yl)(dimethylamino)methylene]-N-methylmetanaminium tetrafluoroborate (TBTU), or benzotriazol-1-yl-oxytripyrrolidinophosphonium hexafluorophosphate (PyBOP), can be employed to form an activated carboxylic acid derivative. Optionally, organic tertiary bases such as triethylamine (TEA) or diisopropylethylamine (DIEA) can also be employed. The formation of the activated carboxylic acid derivative can take place in an inert solvent such as dichloromethane (DCM), N,N-dimethylformamide (DMF), N-methylpyrrolidone (NMP), N,N-dimethylacetamide (DMA, DMAc), or mixtures of any of the foregoing at an appropriate temperature such as from about 0° C. to about 40° C.

Aminolysis of in situ generated or isolated activated carboxylic derivatives with the appropriately functionalized amine derivative (HNR³R⁴) (Scheme 2) can take place in the presence of a suitable base such as an organic tertiary base, i.e., triethylamine (TEA), diethylaminoethylamine (DIEA), pyridine, or mixtures of any of the foregoing, optionally in the presence of suitable additives such as nucleophilic acylation catalysts, i.e., 4-N,N-dimethylaminopyridine (DMAP), and in the same or other inert solvent as used for the activation step such as dichloromethane (DCM), N,N-dimethylformamide (DMF), N-methylpyrrolidone (NMP), N,N-dimethylacetamide (DMA, DMAc), or mixtures of any of the foregoing, at an appropriate temperature such as from about 0° C. to about 70° C.

Functionalized 1-hydroxy acetamides useful for the preparation of MMF acetamide prodrugs of Formula (I) can be also prepared according to Scheme 2:

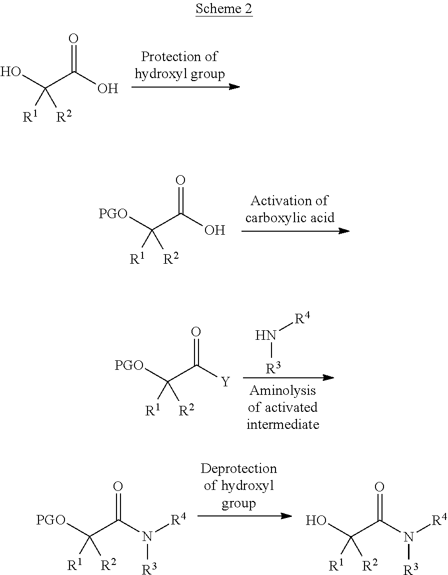

wherein PG is a hydroxyl protecting group; Y is a leaving group such as chloro or an O-isourea derived radical; and R¹, R², R³, and R⁴ are as defined herein.

Certain of the functionalized and activated 1-hydroxy acetic acid derivatives are commercially available, i.e., benzyloxyacetic acid and tert-butyloxy lactic acid. Methods for introducing hydroxyl protecting groups (PGs) are well known in the art. Useful protecting groups to temporarily block the hydroxyl group of functionalized 1-hydroxy acetic acids include certain alkyl such as (substituted) benzyl ethers, tert-butyl ethers, trityl ether, or various silyl ethers such as tert-butyl dimethylsilyl ether, triisopropylsilyl ether, or tert-butyldiphenylsilyl ethers.

Certain protected, functionalized and activated 1-hydroxy acetic acid derivatives are commercially available, i.e., benzyloxyacetyl chloride. Alternatively, the chemical activation of the protected and functionalized 1-hydroxy acetic acid derivative to the corresponding activated carboxylic acid derivative, i.e., carboxylic acid chloride, O-acylisourea, activated esters, etc., can be achieved using similar reaction procedures and conditions as those described in Scheme 1 for the activation of functionalized 1-halo acetic acid derivatives.

Aminolysis of in situ generated or isolated protected, functionalized, and activated 1-hydroxy acetic derivatives with functionalized amines (HNR³R⁴) can take place using similar reaction procedures and conditions as those described in Scheme 1 for the aminolysis of functionalized, protected, and activated 1-halo acetic acid derivatives.

Orthogonal (or ordered) deprotection of the protected 1-hydroxyacetic acid derivative liberates the corresponding free hydroxyl group. Deprotection methods, procedures, and practices are well known in the art.

In certain embodiments, the protecting group can be an alkyl group such as a tert-butyl group. Deprotection may be carried out by contacting a tert-butyl protected functionalized 1-hydroxy acetamide derivative with an excess of a strong Brønsted acid such as trifluoroacetic acid (TFA) or hydrogen chloride (HCl) in an inert solvent such as dichloromethane (DCM), diethyl ether (Et₂O), 1,4-dioxane, or mixtures of any of the foregoing, at an appropriate temperature such as from about 0° C. to about 40° C.

In certain embodiments, the protecting group can be selected from an alkyl group such as a benzyl group. When the protecting group is a benzyl group, deprotection may be carried out by reacting the functionalized 1-hydroxy acetamide derivative with gaseous hydrogen (H₂) in the presence of a heterogenous catalyst, i.e., 5-10 wt-% palladium on activated or wet coal, in a solvent such as methanol (MeOH), ethanol (EtOH), ethyl acetate (EtOAc), or mixtures of any of the foregoing, optionally in the presence of a small amount of an activator such as 1 N aq. hydrochloric acid at an appropriate temperature such as from about 0° C. to about 40° C. and under a hydrogen atmosphere at a pressure of about 15 psi to about 60 psi.

Acetamide MMF prodrugs of Formula (I) can be prepared according to Scheme 3:

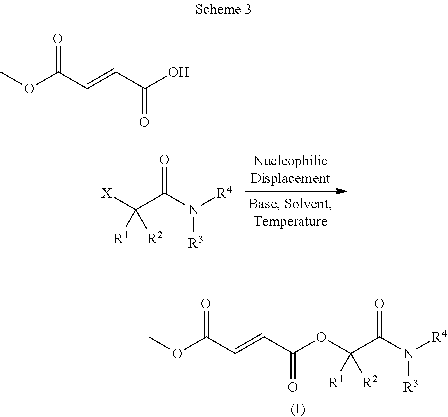

wherein X is a leaving group such as halogen, and R¹, R², R³ and R⁴ are as defined herein. In certain embodiments of Scheme 3, X is chloro.

Nucleophilc displacement of the monoalkyl fumaric acid with the functionalized 1-halo acetamide (Scheme 1) as shown in Scheme 3 can take place in the presence of an inorganic base such as an alkali carbonate such as cesium hydrogencarbonate (CsHCO₃), cesium carbonate (Cs₂CO₃), or potassium carbonate (K₂CO₃). Optionally, organic tertiary bases such as triethylamine (TEA), diisopropylethylamine (DIEA), or amidine; guanidine-based bases such as 1,5-diazabicyclo[4.3.0]non-5-ene (DBN), or 1,8-diazabicyclo[5.4.0]undec-7-ene (DBU), 1,1,3,3-tetramethylguanidine (TMG); silver salts such silver(I) oxide (Ag₂O) or silver(I) carbonate (Ag₂CO₃); or other halide scavengers known in the art can be employed. The corresponding alkali, tri- and tetraalkylammonoium, amidine, or guanide salts of the monoalkyl fumarate can be generated in situ or, alternatively, can be prepared separately. The reaction can take place in an inert solvent such as N,N-dimethylformamide (DMF), N-methylpyrrolidone (NMP), N,N-dimethylacetamide (DMA, DMAc), dimethylsulfoxide (DMSO), tetrahydrofuran (THF), toluene, or mixtures of any of the foregoing at an appropriate temperature such as from about room temperature to about 70° C.

Acetamide MMF prodrugs of Formula (I) can also be prepared according to Scheme 4:

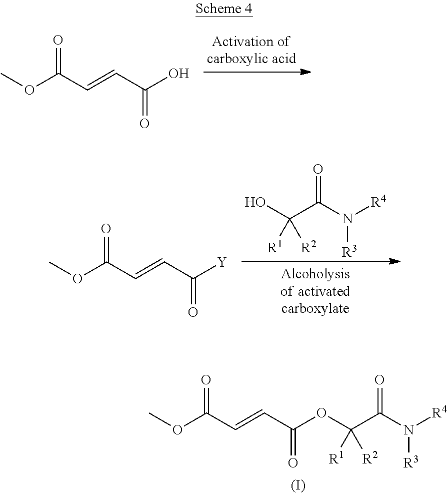

wherein Y is a suitable leaving group such as halogen, an O-acylisourea, various triazolol esters, or others; and R¹, R², R³ and R⁴ are as defined herein. In certain embodiments of Scheme 4, Y is chloro.

Chemical activation of the carboxylic acid to the corresponding carboxylic acid chloride as shown in Scheme 4 can be accomplished by reaction with a chlorination agent such as thionyl chloride (SOCl₂), oxalyl chloride (C₂O₂Cl₂), phosphorous pentachloride (PCl₅), or others, optionally in the presence of a catalyst such as N,N-dimethylformamide (DMF), and either in substance (absence of solvent) or in an inert organic solvent such as dichloromethane (DCM) at an appropriate temperature such as from about 0° C. to about 70° C. Chemical activation of the carboxylic acid as shown in Scheme 4 can be performed in situ without isolating the activated substrate prior to the subsequent alcoholysis step. Optionally, the activated carboxylic acid chloride can be isolated and/or purified using methods well known in the art, i.e. fractional distillation.

Alternatively, carbodiimide dehydration agents such as N,N′-diisopropylcarbodiimide (DIC), N,N′-dicyclohexylcarbodiimide (DCC), or 1-ethyl-3-(3-dimethylaminopropyl) carbodiimide (EDAC, EDC), optionally in the presence of a catalytic or stoichiometric amount of an additive such as 4-(N,N-dimethylaminopyridine (DMAP) (Steglich esterification conditions), 1-hydroxybenzotriazole (HOBt), 1-hydroxy-7-aza-benzotriazole (HOAt), or N-hydroxysuccinimide (HOSu); a uronium or phosphonium salt with non-nucleophilic anions such as N-[(1H-benzotriazol-1-yl)(dimethylamino)methylene]-N-methylmetanaminium hexafluorophosphate (HBTU), N-[(dimethylamino)-1H-1,2,3-triazolo[4,5-b]pyridin-1-ylmethylene]-N-methylmetanaminium hexafluorophosphate N-oxide (HATU), N-[(1H-benzotriazol-1-yl)(dimethylamino)methylene]-N-methylmetanaminium tetrafluoroborate (TBTU), or benzotriazol-1-yl-oxytripyrrolidinophosphonium hexafluorophosphate (PyBOP), can be employed to form an activated monoalkyl fumarate derivative. Optionally, organic tertiary bases such as triethylamine (TEA) or diethylaminoethylamine (DIEA) can also be employed. The formation of activated monoalkyl fumarate derivatives can take place in an inert solvent such as dichloromethane (DCM), N,N-dimethylformamide (DMF), N-methylpyrrolidone (NMP), N,N-dimethylacetamide (DMA, DMAc), or mixtures of any of the foregoing at an appropriate temperature such as from about room temperature to about 70° C.

Alcoholysis of the activated monoalkyl fumarate derivative with a functionalized hydroxy acetamide derivative (Scheme 2) can take place in the presence of a base, for example, an organic tertiary base such as, triethylamine (TEA), diethylaminoethylamine (DIEA), or pyridine, optionally in the presence of an additive such as a nucleophilic acylation catalyst, i.e., 4-(N,N-dimethylaminopyridine (DMAP) (Steglich esterification conditions), and in the same or other inert solvent as used for the activation step such as dichloromethane (DCM), N,N-dimethylformamide (DMF), N-methylpyrrolidone (NMP), N,N-dimethylacetamide (DMA, DMAc), or mixtures of any of the foregoing at an appropriate temperature such as from about 0° C. to about 70° C.

Acetamide MMF prodrugs of Formula (I) can also be prepared according to Scheme 5:

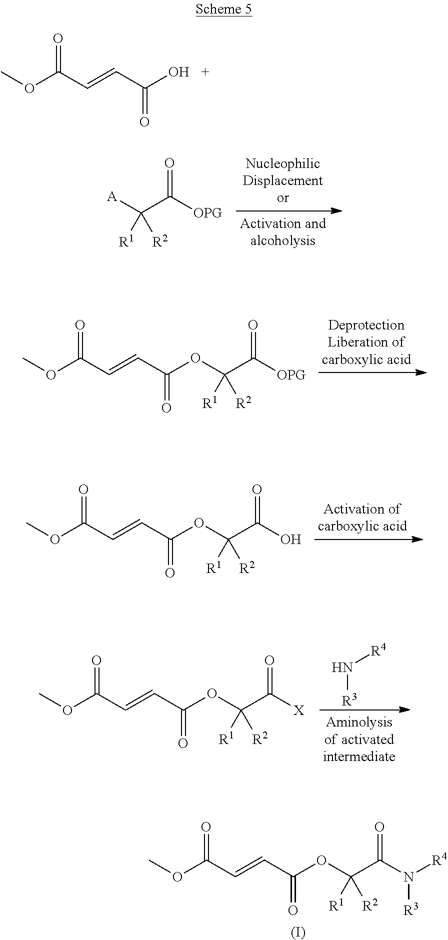

wherein A is either a leaving group such as halogen or a nucleophilic coupling group such as hydroxyl; Y is a leaving group such as halogen, an O-acylisourea, various triazolol esters, or others; PG is a carboxyl protecting group; and R¹, R², R³ and R⁴ are as defined herein. In certain embodiments of Scheme 5, X is bromo, PG is tert-butyl, each of R¹ and R² is hydrogen, and the electrophile is tert-butyl bromoacetate. In certain embodiments of Scheme 5, Y is chloro or O-acylisourea derived from 1-ethyl-3-(3-dimethylaminopropyl)carbodiimide (EDAC).

The nucleophilc displacement reaction of the monoalkyl fumaric acid with a protected and functionalized 1-halo acetic acid derivative, i.e., commercially available tert-butyl bromoacetate or others, can take place using similar reaction procedures and conditions as those described in Scheme 3 for the direct formation of functionalized acetamide MMF prodrugs of Formula (I) from a monoalkyl fumaric acid and an appropriately functionalized 1-halo acetamide.

Alcoholysis of an intermediate activated monoalkyl fumaric acid derivative and a protected and functionalized 1-hydroxy acetic acid derivative can take place using similar reaction procedures and conditions as those used in Scheme 4 for the direct formation of functionalized acetamide MMF prodrugs of Formula (I) from a monoalkyl fumaric acid and an appropriately functionalized 1-hydroxy acetamide.

Orthogonal (or ordered) deprotection of a protected monoalkyl fumaric acid-functionalized acetic acid derivative liberates the corresponding free monoalkyl fumarate ester intermediate bearing a free carboxylic acid moiety. When the protecting group is a tert-butyl group, deprotection may be carried out by contacting the tert-butyl protected fumaric acid derivative with an excess of a strong Brønsted acid such as trifluoroacetic acid (TFA) or hydrogen chloride (HCl) in an inert solvent such as dichloromethane (DCM), diethyl ether (Et₂O), 1,4-dioxane, or mixtures of any of the foregoing, at an appropriate temperature such as from about 0° C. to about 40° C.

Chemical activation of the liberated monoalkyl fumarate-functionalized hydroxyacetic derivative (carboxylic acid) to the corresponding activated carboxylic acid derivative, i.e., carboxylic acid chloride, O-acylisourea, activated esters, etc., can be accomplished using reaction procedures and conditions similar to those described in Scheme 4 for the activation of monoalkyl fumaric acid direct formation of functionalized acetamide MMF prodrugs of Formula (I) from the monoalkyl fumaric acid and the corresponding functionalized hydroxyl acetamide.

Aminolysis of in situ generated or isolated activated monoalkyl fumarate functionalized hydroxyacetic derivatives with functionalized amines (HNR³R⁴) can take place using reaction procedures and conditions similar to those described in Schemes 1 and 2 for the aminolysis of protected, suitably functionalized and activated hydroxy acetic acid derivatives.

Certain of the functionalized 1-haloalkyl carboxylates (1-acyloxyalkyl halides) or functionalized 1-alkoxycarbonyloxyalkyl halides useful for preparing compounds of Formula (II) are available from commercial sources. Non-commercially available 1-haloalkyl carboxylates (1-acyloxyalkyl halides) or functionalized 1-alkoxycarbonyloxyalkyl halides can be prepared by methods well known in the art and are briefly described in Schemes 6 and 7.

1-Acyloxyalky halides useful for the preparation of MMF prodrugs of Formula (II) can be prepared according to Scheme 6:

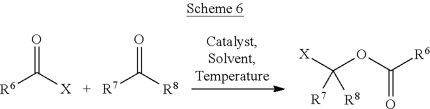

wherein X is a leaving group such as halogen; and R⁶, R⁷, R⁸ are as defined herein. In certain embodiments of Scheme 6, X is chloro and R⁶ is 2-[methyl (2E)but-2-ene-4-ate]yl; one of R⁷ and R⁸ is hydrogen and the other of R⁷ and R⁸ is alkyl.

Functionalized 1-haloalkyl carboxylates (1-acyloxyalkyl halides) may be prepared by contacting a functionalized carboxylic acid halide such as a carboxylic acid chloride with a functionalized carbonyl compound such as an aldehyde in the presence of a Lewis acid catalyst such as anhydrous zinc chloride (ZnCl₂) in an inert solvent such as dichloromethane (DCM) at a temperature from about −10° C. to room temperature. The 1-chloroalkyl carboxylates (1-acyloxyalkyl chlorides) may be used directly or may be isolated and purified by methods well known in the art such as by fractional distillation or silica gel column chromatography.

1-Alkoxy- and 1-aryloxycarbonyloxyalkyl halides useful for the preparation of MMF prodrugs of Formula (II) can be prepared according to Scheme 7:

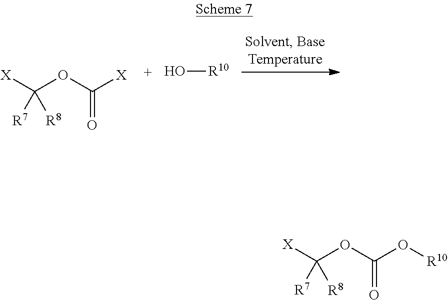

wherein X is a leaving group such as halogen, and R⁷, R⁸, and R¹⁰ are as defined herein. In certain embodiments of Scheme 7, X is chloro and R¹⁰ together with the oxygen atom to which it is bonded is equivalent to R⁶, which is defined herein.

Functionalized 1-alkoxy- or aryloxycarbonyloxyalkyl halides may be prepared by contacting a functionalized haloalkyl halo formate such as a functionalized chloro alkyl- or aryl chloroformate with a functionalized alcohol or phenol (HOR¹⁰) in the presence of a base such as an organic secondary and tertiary base, i.e., dicyclohexyl amine (DCHA), triethylamine (TEA), diisopropylethylamine (DIEA, Hünigs-base), pyridine, in an inert solvent such as dichloromethane (DCM) at a temperature from about −10° C. to room temperature. The 1-alkoxy- or aryloxycarbonyloxyalkyl halides may be used directly or may be isolated and purified by methods well known in the art such as by fractional distillation or silica gel column chromatography.

Acyloxyalkyl and alkoxycarbonyloxyalkyl MMF prodrugs of Formula (II) can be prepared according to Scheme 8:

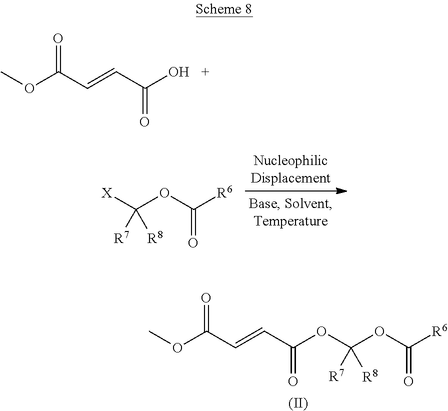

wherein X is a leaving group such as halogen, and R⁶, R⁷ and R⁸ are as defined herein.

Nucleophilic displacement of the monoalkyl fumaric acid with a functionalized 1-halo (Scheme 1) as shown in Scheme 8 can take place in the presence of an inorganic base such as an alkali carbonate, i.e., cesium bicarbonate (CsHCO₃), cesium carbonate (Cs₂CO₃), or potassium carbonate (K₂CO₃). Alternatively, organic secondary and tertiary bases such as dicyclohexyl amine (DCHA), triethylamine (TEA), diisopropylethylamine (DIEA), amidine or guanidine-based bases such as 1,5-diazabicyclo[4.3.0]non-5-ene (DBN), 1,8-diazabicyclo[5.4.0]undec-7-ene (DBU), or 1,1,3,3-tetramethylguanidine (TMG); silver salts such silver(I) oxide (Ag₂O) or silver(I) carbonate (Ag₂CO₃); or other halide scavengers known in the art can be employed. The corresponding alkali, tri- and tetraalkylammonoium, amidine, or guanide salts of the monoalkyl fumarate can be generated in situ or, alternatively, can be prepared separately. The reaction can take place in an inert solvent such as N,N-dimethylformamide (DMF), N-methylpyrrolidone (NMP), N,N-dimethylacetamide (DMA, DMAc), dimethylsulfoxide (DMSO), or tetrahydrofuran (THF), toluene, or mixtures of any of the foregoing at an appropriate temperature such as from about room temperature to about 70° C.

The coupling of fumaric acid or monoalkyl fumarates, i.e., mono tert-butyl fumarate, with functionalized 1-halo acetamide derivatives, functionalized 1-haloalkyl carboxylates (1-acyloxyalkyl halides), or 1-alkoxy- or aryloxycarbonyloxyalkyl halides, can take place using reaction procedures and conditions similar to those described in Schemes 3 and 8 for the direct formation of functionalized acetamide MMF prodrugs of Formula (I) (Scheme 3) or acyloxyalkyl or alkoxy-aryloxycarbonyloxyalkyl MMF prodrugs of Formula (II) (Scheme 8).

In certain embodiments, orthogonal (or ordered) deprotection (or liberation of the free carboxylic acid) from the corresponding functionalized acetamide or acyloxyalkyl or alkoxy-/aryloxycarbonyloxyalkyl tert-butyl fumarates may be accomplished using reaction procedures and conditions similar to those described in Scheme 5.

### Pharmaceutical Compositions

Pharmaceutical compositions provided by the present disclosure may comprise a therapeutically effective amount of DMF and/or a compound of Formulae (I)-(V) together with a suitable amount of one or more pharmaceutically acceptable vehicles so as to provide a composition for proper administration to a patient. Suitable pharmaceutical vehicles are described in the art.

In certain embodiments, DMF and/or or a compound of Formulae (I), (II) and (V) may be incorporated into pharmaceutical compositions to be administered orally. Oral administration of such pharmaceutical compositions may result in uptake of DMF and/or a compound of Formulae (I), (II) and (V) throughout the intestine and entry into the systemic circulation. Such oral compositions may be prepared in a manner known in the pharmaceutical art and comprise DMF and/or a compound of Formulae (I), (II) and (V) and at least one pharmaceutically acceptable vehicle. Oral pharmaceutical compositions may include a therapeutically effective amount of DMF and/or a compound of Formulae (I), (II) and (V) and a suitable amount of a pharmaceutically acceptable vehicle, so as to provide an appropriate form for administration to a patient.

DMF and/or a compound of Formulae (I), (II) and (V) may be incorporated into pharmaceutical compositions to be administered by any other appropriate route of administration including intradermal, intramuscular, intraperitoneal, intravenous, subcutaneous, intranasal, epidural, oral, sublingual, intracerebral, intravaginal, transdermal, rectal, inhalation, or topical.

Pharmaceutical compositions comprising DMF and/or a compound of Formulae (I), (II) and (V) and may be manufactured by means of conventional mixing, dissolving, granulating, dragee-making, levigating, emulsifying, encapsulating, entrapping, or lyophilizing processes. Pharmaceutical compositions may be formulated in a conventional manner using one or more physiologically acceptable carriers, diluents, excipients, or auxiliaries, which facilitate processing of DMF and/or a compound of Formulae (I), (II) and (V) or crystalline forms thereof and one or more pharmaceutically acceptable vehicles into formulations that can be used pharmaceutically. Proper formulation is dependent upon the route of administration chosen. Pharmaceutical compositions provided by the present disclosure may take the form of solutions, suspensions, emulsion, tablets, pills, pellets, capsules, capsules containing liquids, powders, sustained-release formulations, suppositories, emulsions, aerosols, sprays, suspensions, or any other form suitable for administration to a patient.

Pharmaceutical compositions provided by the present disclosure may be formulated in a unit dosage form. A unit dosage form refers to a physically discrete unit suitable as a unitary dose for patients undergoing treatment, with each unit containing a predetermined quantity of DMF and/or a compound of Formulae (I), (II) and (V) calculated to produce an intended therapeutic effect. A unit dosage form may be for a single daily dose, for administration 2 times per day, or one of multiple daily doses, e.g., 3 or more times per day. When multiple daily doses are used, a unit dosage form may be the same or different for each dose. One or more dosage forms may comprise a dose, which may be administered to a patient at a single point in time or during a time interval.

Pharmaceutical compositions comprising DMF and/or a compound of Formulae (I), (II) and (V) may be formulated for immediate release.

In certain embodiments, an oral dosage form provided by the present disclosure may be a controlled release dosage form. Controlled delivery technologies can improve the absorption of a drug in a particular region or regions of the gastrointestinal tract. Controlled drug delivery systems may be designed to deliver a drug in such a way that the drug level is maintained within a therapeutically effective window and effective and safe blood levels are maintained for a period as long as the system continues to deliver the drug with a particular release profile in the gastrointestinal tract. Controlled drug delivery may produce substantially constant blood levels of a drug over a period of time as compared to fluctuations observed with immediate release dosage forms. For some drugs, maintaining a constant blood and tissue concentration throughout the course of therapy is the most desirable mode of treatment. Immediate release of drugs may cause blood levels to peak above the level required to elicit a desired response, which may waste the drug and may cause or exacerbate toxic side effects. Controlled drug delivery can result in optimum therapy, and not only can reduce the frequency of dosing, but may also reduce the severity of side effects. Examples of controlled release dosage forms include dissolution controlled systems, diffusion controlled systems, ion exchange resins, osmotically controlled systems, erodable matrix systems, pH independent formulations, gastric retention systems, and the like.

An appropriate oral dosage form for a particular pharmaceutical composition provided by the present disclosure may depend, at least in part, on the gastrointestinal absorption properties of DMF and/or a compound of Formulae (I), (II) and (V) the stability of DMF and/or a compound of Formulae (I), (II) and (V) in the gastrointestinal tract, the pharmacokinetics of DMF and/or a compound of Formulae (I), (II) and (V) and the intended therapeutic profile. An appropriate controlled release oral dosage form may be selected for a particular MMF prodrug. For example, gastric retention oral dosage forms may be appropriate for compounds absorbed primarily from the upper gastrointestinal tract, and sustained release oral dosage forms may be appropriate for compounds absorbed primarily from the lower gastrointestinal tract. Certain compounds are absorbed primarily from the small intestine. In general, compounds traverse the length of the small intestine in about 3 to 5 hours. For compounds that are not easily absorbed by the small intestine or that do not dissolve readily, the window for active agent absorption in the small intestine may be too short to provide a desired therapeutic effect.

In certain embodiments, pharmaceutical compositions provided by the present disclosure may be practiced with dosage forms adapted to provide sustained release of DMF and/or a compound of Formulae (I), (II) and (V) upon oral administration. Sustained release oral dosage forms may be used to release drugs over a prolonged time period and are useful when it is desired that a drug or drug form be delivered to the lower gastrointestinal tract, including the colon. Sustained release oral dosage forms include any oral dosage form that maintains therapeutic concentrations of a drug in a biological fluid such as the plasma, blood, cerebrospinal fluid, or in a tissue or organ for a prolonged time period. Sustained release oral dosage forms include diffusion-controlled systems such as reservoir devices and matrix devices, dissolution-controlled systems, osmotic systems, and erosion-controlled systems. Sustained release oral dosage forms and methods of preparing the same are well known in the art.

An appropriate dose of DMF and/or a compound of Formulae (I), (II) and (V) or pharmaceutical composition comprising DMF and/or a compound of Formulae (I), (II) and (V) may be determined according to any one of several well-established protocols. For example, animal studies such as studies using mice, rats, dogs, and/or monkeys may be used to determine an appropriate dose of a pharmaceutical compound. Results from animal studies may be extrapolated to determine doses for use in other species, such as for example, humans.

### Uses

DMF and compounds of Formulae (I), (II) and (V) are prodrugs of MMF. Thus, DMF, compounds of Formulae (I), (II) and (V), other MMF prodrugs and pharmaceutical compositions thereof may be administered to a patient suffering from a disease chosen from adrenal leukodystrophy, Alexanders Disease, Alpers' Disease, balo concentric sclerosis, bronchiolitis obliterans organizing pneumonia, Canavan disease, central nervous system vasculitis, Charcott-Marie-Tooth Disease, childhood ataxia with central nervous system hypomyelination, chronic inflammatory demyelinating polyneuropathy, cutaneous lupus erythematosus, chronic lymphocytic leukemia, diabetic retinopathy, globoid cell leukodystrophy, graft versus host disease, hepatitis C viral infection, herpes simplex viral infection, human immunodeficiency viral infection, lichen planus, macular degeneration, monomelic amyotrophy, necrobiosis lipoidosis, neurodegeneration with brain iron accumulation, neuromyelitis optica, neurosarcoidosis, optic neuritis, pareneoplastic syndromes, Pelizaeus-Merzbacher disease, primary lateral sclerosis, progressive supranuclear palsy, Schilder's Disease, subacute necrotizing myelopathy, Susac syndrome, transverse myelitis, a tumor and Zellweger syndrome.

In some embodiments, DMF, compounds of Formulae (I), (II) and (V), other MMF prodrugs and pharmaceutical compositions thereof may be administered to a patient suffering from adrenal leukodystrophy. In some embodiments, DMF, compounds of Formulae (I), (II) and (V), other MMF prodrugs and pharmaceutical compositions thereof may be administered to a patient suffering from Alexanders Disease. In some embodiments, DMF, compounds of Formulae (I), (II) and (V), other MMF prodrugs and pharmaceutical compositions thereof may be administered to a patient suffering from Alpers' Disease. In some embodiments, DMF, compounds of Formulae (I), (II) and (V), other MMF prodrugs and pharmaceutical compositions thereof may be administered to a patient suffering from balo concentric sclerosis. In some embodiments, DMF, compounds of Formulae (I), (II) and (V), other MMF prodrugs and pharmaceutical compositions thereof may be administered to a patient suffering from bronchiolitis obliterans organizing pneumonia. In some embodiments, DMF, compounds of Formulae (I), (II) and (V), other MMF prodrugs and pharmaceutical compositions thereof may be administered to a patient suffering from Canavan disease. In some embodiments, DMF, compounds of Formulae (I), (II) and (V), other MMF prodrugs and pharmaceutical compositions thereof may be administered to a patient suffering from central nervous system vasculitis. In some embodiments, DMF, compounds of Formulae (I), (II) and (V), other MMF prodrugs and pharmaceutical compositions thereof may be administered to a patient suffering from Charcott-Marie-Tooth Disease. In some embodiments, DMF, compounds of Formulae (I), (II) and (V), other MMF prodrugs and pharmaceutical compositions thereof may be administered to a patient suffering from childhood ataxia with central nervous system hypomyelination. In some embodiments, DMF, compounds of Formulae (I), (II) and (V), other MMF prodrugs and pharmaceutical compositions thereof may be administered to a patient suffering from chronic inflammatory demyelinating polyneuropathy. In some embodiments, DMF, compounds of Formulae (I), (II) and (V), other MMF prodrugs and pharmaceutical compositions thereof may be administered to a patient suffering from cutaneous lupus erythematosus. In some embodiments, DMF, compounds of Formulae (I), (II) and (V), other MMF prodrugs and pharmaceutical compositions thereof may be administered to a patient suffering from chronic lymphocytic leukemia. In some embodiments, DMF, compounds of Formulae (I),

(II) and (V), other MMF prodrugs and pharmaceutical compositions thereof may be administered to a patient suffering from diabetic retinopathy. In some embodiments, DMF, compounds of Formulae (I), (II) and (V), other MMF prodrugs and pharmaceutical compositions thereof may be administered to a patient suffering from globoid cell leukodystrophy. In some embodiments, DMF, compounds of Formulae (I), (II) and (V), other MMF prodrugs and pharmaceutical compositions thereof may be administered to a patient suffering from graft versus host disease. In some embodiments, DMF, compounds of Formulae (I), (II) and (V), other MMF prodrugs and pharmaceutical compositions thereof may be administered to a patient suffering from hepatitis C viral infection. In some embodiments, DMF, compounds of Formulae (I), (II) and (V), other MMF prodrugs and pharmaceutical compositions thereof may be administered to a patient suffering from herpes simplex viral infection. In some embodiments, DMF, compounds of Formulae (I), (II) and (V), other MMF prodrugs and pharmaceutical compositions thereof may be administered to a patient suffering from human immunodeficiency viral infection. In some embodiments, DMF, compounds of Formulae (I), (II) and (V), other MMF prodrugs and pharmaceutical compositions thereof may be administered to a patient suffering from lichen planus. In some embodiments, DMF, compounds of Formulae (I), (II) and (V), other MMF prodrugs and pharmaceutical compositions thereof may be administered to a patient suffering from macular degeneration. In some embodiments, DMF, compounds of Formulae (I), (II) and (V), other MMF prodrugs and pharmaceutical compositions thereof may be administered to a patient suffering from monomelic amyotrophy. In some embodiments, DMF, compounds of Formulae (I), (II) and (V), other MMF prodrugs and pharmaceutical compositions thereof may be administered to a patient suffering from necrobiosis lipoidosis. In some embodiments, DMF, compounds of Formulae (I), (II) and (V), other MMF prodrugs and pharmaceutical compositions thereof may be administered to a patient suffering from neurodegeneration with brain iron accumulation. In some embodiments, DMF, compounds of Formulae (I), (II) and (V), other MMF prodrugs and pharmaceutical compositions thereof may be administered to a patient suffering from neuromyelitis optica. In some embodiments, DMF, compounds of Formulae (I), (II) and (V), other MMF prodrugs and pharmaceutical compositions thereof may be administered to a patient suffering from neurosarcoidosis. In some embodiments, DMF, compounds of Formulae (I), (II) and (V), other MMF prodrugs and pharmaceutical compositions thereof may be administered to a patient suffering from optic neuritis. In some embodiments, DMF, compounds of Formulae (I), (II) and (V), other MMF prodrugs and pharmaceutical compositions thereof may be administered to a patient suffering from pareneoplastic syndromes. In some embodiments, DMF, compounds of Formulae (I), (II) and (V), other MMF prodrugs and pharmaceutical compositions thereof may be administered to a patient suffering from Pelizaeus-Merzbacher disease. In some embodiments, DMF, compounds of Formulae (I), (II) and (V), other MMF prodrugs and pharmaceutical compositions thereof may be administered to a patient suffering from primary lateral sclerosis. In some embodiments, DMF, compounds of Formulae (I), (II) and (V), other MMF prodrugs and pharmaceutical compositions thereof may be administered to a patient suffering from progressive supranuclear palsy. In some embodiments, DMF, compounds of Formulae (I), (II) and (V), other MMF prodrugs and pharmaceutical compositions thereof may be administered to a patient suffering from Schilder's Disease. In some embodiments, DMF, compounds of Formulae (I), (II) and (V), other MMF prodrugs and pharmaceutical compositions thereof may be administered to a patient suffering from subacute necrotizing myelopathy. In some embodiments, DMF, compounds of Formulae (I), (II) and (V), other MMF prodrugs and pharmaceutical compositions thereof may be administered to a patient suffering from Susac syndrome. In some embodiments, DMF, compounds of Formulae (I), (II) and (V), other MMF prodrugs and pharmaceutical compositions thereof may be administered to a patient suffering from transverse myelitis. In some embodiments, DMF, compounds of Formulae (I), (II) and (V), other MMF prodrugs and pharmaceutical compositions thereof may be administered to a patient suffering from a tumor. In some embodiments, DMF, compounds of Formulae (I), (II) and (V), other MMF prodrugs and pharmaceutical compositions thereof may be administered to a patient suffering from Zellweger syndrome.

Methods of treating a disease in a patient provided by the present disclosure comprise administering to a patient in need of such treatment a therapeutically effective amount of a MMF prodrug, such as DMF and/or a compound of Formulae (I), (II) and (V). These MMF prodrugs, and pharmaceutical compositions thereof, provide therapeutic or prophylactic plasma and/or blood concentrations of MMF following administration to a patient.

MMF prodrugs such as DMF and the compounds of Formulae (I), (II) and (V) may be included in a pharmaceutical composition and/or dosage form adapted for oral administration, although an MMF prodrug of Formulae (I), (II) and (V) may also be administered by any other appropriate route, such as for example, by injection, infusion, inhalation, transdermal, or absorption through epithelial or mucosal membranes (e.g., oral, rectal, and/or intestinal mucosa).

MMF prodrugs such as DMF and/or a compound of Formulae (I), (II) and (V) may be administered in an amount and using a dosing schedule as appropriate for treatment of a particular disease. Daily doses of a MMF prodrug may range from about 0.01 mg/kg to about 50 mg/kg, from about 0.1 mg/kg to about 50 mg/kg, from about 1 mg/kg to about 50 mg/kg, and in certain embodiments, from about 5 mg/kg to about 25 mg/kg. In certain embodiments, MMF prodrugs may be administered at a dose over time from about 1 mg to about 5 g per day, from about 10 mg to about 4 g per day, and in certain embodiments from about 20 mg to about 2 g per day. An appropriate dose of a MMF prodrug may be determined based on several factors, including, for example, the body weight and/or condition of the patient being treated, the severity of the disease being treated, the incidence and/or severity of side effects, the manner of administration, and the judgment of the prescribing physician. Appropriate dose ranges may be determined by methods known to those skilled in the art.

MMF prodrugs such as DMF and the compounds of Formulae (I), (II) and (V) may be assayed in vitro and in vivo for the desired therapeutic or prophylactic activity prior to use in humans. In vivo assays, for example using appropriate animal models, may also be used to determine whether administration of a MMF prodrug is therapeutically effective.

In certain embodiments, a therapeutically effective dose of a MMF prodrug such as DMF and/or a compound of Formulae (I), (II) and (V) may provide therapeutic benefit without causing substantial toxicity including adverse side effects. Toxicity of MMF prodrugs and/or metabolites thereof may be determined using standard pharmaceutical procedures and may be ascertained by those skilled in the art. The dose ratio between toxic and therapeutic effect is the therapeutic index. A dose of a MMF prodrug may be within a range capable of establishing and maintaining a therapeutically effective circulating plasma and/or blood concentration of a MMF prodrug that exhibits little or no toxicity.

MMF prodrugs such as DMF and/or a compound of Formulae (I), (II) and (V) may be used to treat a disease chosen from adrenal leukodystrophy, Alexanders Disease, Alpers' Disease, balo concentric sclerosis, bronchiolitis obliterans organizing pneumonia, Canavan disease, central nervous system vasculitis, Charcott-Marie-Tooth Disease, childhood ataxia with central nervous system hypomyelination, chronic inflammatory demyelinating polyneuropathy, cutaneous lupus erythematosus, chronic lymphocytic leukemia, diabetic retinopathy, globoid cell leukodystrophy, graft versus host disease, hepatitis C viral infection, herpes simplex viral infection, human immunodeficiency viral infection, lichen planus, macular degeneration, monomelic amyotrophy, necrobiosis lipoidosis, neurodegeneration with brain iron accumulation, neuromyelitis optica, neurosarcoidosis, optic neuritis, pareneoplastic syndromes, Pelizaeus-Merzbacher disease, primary lateral sclerosis, progressive supranuclear palsy, Schilder's Disease, subacute necrotizing myelopathy, Susac syndrome, transverse myelitis, a tumor and Zellweger syndrome. The underlying etiology of any of the foregoing diseases being treated may have a multiplicity of origins. Further, in certain embodiments, a therapeutically effective amount of the MMF prodrug may be administered to a patient, such as a human, as a preventative measure against the foregoing diseases and disorders. Thus, a therapeutically effective amount of DMF and/or a compound of Formulae (I), (II) and (V) may be administered as a preventative measure to a patient having a predisposition for and/or history of adrenal leukodystrophy, Alexanders Disease, Alpers' Disease, balo concentric sclerosis, bronchiolitis obliterans organizing pneumonia, Canavan disease, central nervous system vasculitis, Charcott-Marie-Tooth Disease, childhood ataxia with central nervous system hypomyelination, chronic inflammatory demyelinating polyneuropathy, cutaneous lupus erythematosus, chronic lymphocytic leukemia, diabetic retinopathy, globoid cell leukodystrophy, graft versus host disease, hepatitis C viral infection, herpes simplex viral infection, human immunodeficiency viral infection, lichen planus, macular degeneration, monomelic amyotrophy, necrobiosis lipoidosis, neurodegeneration with brain iron accumulation, neuromyelitis optica, neurosarcoidosis, optic neuritis, pareneoplastic syndromes, Pelizaeus-Merzbacher disease, primary lateral sclerosis, progressive supranuclear palsy, Schilder's Disease, subacute necrotizing myelopathy, Susac syndrome, transverse myelitis, a tumor and/or Zellweger syndrome.

### Adrenal Leukodystrophy

Adrenal leukodystrophy (which is also sometimes referred to as adrenoleukodystrophy) describes several closely related inherited disorders that disrupt the breakdown (metabolism) of certain fats (very-long-chain fatty acids). Adrenal leukodystrophy is passed down from parents to their children as an X-linked genetic trait. It therefore affects mostly males, although some women who are carriers can have milder forms of the disease. It affects approximately 1 in 20,000 people from all races. The condition results in the buildup of very-long-chain fatty acids in the nervous system, adrenal gland, and testes, which disrupts normal activity. The disease is closely related to Schilder's disease, marked by diffuse abnormality of the cerebral white matter and adrenal atrophy. The disease is characterized by mental deterioration progressing to dementia, and by aphasia, apraxia, dysarthria, and loss of vision in about a third of the patients. Almost all patients show abnormal adrenal functioning when tested.

Currently, adrenal leukodystrophy is treated with steroids such as cortisol, eating a diet low in very-long-chain fatty acids and taking Lorenzo's oil, which can lower the blood levels of very-long-chain fatty acids. Bone marrow transplants are also being tested as an experimental treatment.

The efficacy of MMF and MMF prodrugs for treating adrenal leukodystrophy can be determined using animal models and in clinical trials. Suitable animal models for adrenal leukodystrophy are disclosed for example in Fourcade, S., et al. (2010) Hum Mol Genet 19(10), 2005-14; and Khan, M., et al. (2008) J Neurochem 106(4), 1766-79.

### Alexanders Disease

Alexanders disease (which is also sometimes referred to as Alexander disease) is one of a group of neurological conditions known as the leukodystrophies, disorders that are the result of abnormalities in myelin, the “white matter” that protects nerve fibers in the brain. Alexanders disease is a progressive and usually fatal disease. The destruction of white matter is accompanied by the formation of Rosenthal fibers, which are abnormal clumps of protein that accumulate in non-neuronal cells of the brain called astrocytes. Rosenthal fibers are sometimes found in other disorders, but not in the same amount or area of the brain that are featured in Alexanders disease. The infantile form is the most common type of Alexanders disease. It has an onset during the first two years of life. Usually there are both mental and physical developmental delays, followed by the loss of developmental milestones, an abnormal increase in head size, and seizures. The juvenile form of Alexanders disease is less common and has an onset between the ages of two and thirteen. These children may have excessive vomiting, difficulty swallowing and speaking, poor coordination, and loss of motor control. Adult-onset forms of Alexanders disease are rare, but have been reported. The symptoms sometimes mimic those of Parkinson's disease or multiple sclerosis. The disease occurs in both males and females, and there are no ethnic, racial, geographic, or cultural/economic differences in its distribution.

The efficacy of MMF and MMF prodrugs for treating Alexanders Disease can be determined using animal models and in clinical trials. Suitable animal models for Alexanders Disease are disclosed for example in Wang, L., et al. (2011) J Neurosci 31(8), 2868-77.

### Alpers' Disease

Alpers' disease (which is also referred to as Christensen-Krabbe disease, poliodystrophia cerebri, and progressive cerebral or progressive infantile poliodystrophy) is a rare disease of young children, characterized by neuronal degeneration of the cerebral cortex and elsewhere, accompanied by progressive mental deterioration, motor disturbances, seizures, and early death. The disease is a progressive, neurodevelopmental, mitochondrial DNA depletion syndrome characterized by three co-occurring clinical symptoms: psychomotor regression (dementia); seizures; and liver disease. It is an autosomal recessive disease caused by mutation in the gene for the mitochondrial DNA polymerase POLG. The disease occurs in about one in 100,000 persons. Most individuals with Alpers' disease do not show symptoms at birth and develop normally for weeks to years before the onset of symptoms. Diagnosis is established by testing for the POLG gene. Symptoms typically occur months before tissue samples show the mitochondrial DNA depletion, so that these depletion studies cannot be used for early diagnosis. About 80 percent of individuals with Alpers' disease develop symptoms in the first two years of life, and 20 percent develop symptoms between ages 2 and 25. The first symptoms of the disorder are usually nonspecific and may include hypoglycemia secondary to underlying liver disease, failure to thrive, infection-associated encephalopathy, spasticity, myoclonus (involuntary jerking of a muscle or group of muscles), seizures, or liver failure. An increased protein level is seen in cerebrospinal fluid analysis. Cortical blindness (loss of vision due to damage to the area of the cortex that controls vision) develops in about 25 percent of cases. Gastrointestinal dysfunction and cardiomyopathy may occur. Dementia is typically episodic and often associated with an infection that occurs while another disease is in process. Seizures may be difficult to control and unrelenting seizures can cause developmental regression as well. “Alpers-like” disorders without liver disease are genetically different and have a different clinical course. Fewer than one-third of individuals with the “Alpers-like” phenotype without liver disease have POLG mutations.

The efficacy of MMF and MMF prodrugs for treating Alper's Disease can be determined using animal models and in clinical trials.

### Balo Concentric Sclerosis

Balo concentric sclerosis (also sometimes referred to as Balo's disease, encephalitis periaxialis concentrica, leukoencephalitis periaxialis concentrica, and concentric sclerosis) is an atypical form of Schilder's disease in which the demyelination is arranged in concentric rings around a central circle.

The efficacy of MMF and MMF prodrugs for treating balo concentric sclerosis can be determined using animal models and in clinical trials.

### Bronchiolitis Obliterans Organizing Pneumonia

Bronchiolitis obliterans organizing pneumonia (also sometimes referred to as BOOP and/or cryptogenic organizing pneumonia) is a non-infectious pneumonia; specifically, an inflammation of the bronchioles (bronchiolitis) and surrounding tissue in the lungs. It is often a complication of an existing chronic inflammatory disease such as rheumatoid arthritis, or it can be a side effect of certain medications such as amiodarone.

The efficacy of MMF and MMF prodrugs for treating bronchiolitis obliterans organizing pneumonia can be determined using animal models and in clinical trials. Suitable animal models for bronchiolitis obliterans organizing pneumonia are disclosed for example in Majeski et al., Respiratory reovirus 1/L induction of intraluminal fibrosis, a model of bronchiolitis obliterans organizing pneumonia, is dependent on T lymphocytes, Am J Pathol. (2003 October), 163(4):1467-79; and Gillen et al., Rapamycin blocks fibrocyte migration and attenuates bronchiolitis obliterans in a murine model Ann Thorac Surg. (2013 May), 95(5):1768-75.

### Canavan Disease

Canavan disease, one of the most common cerebral degenerative diseases of infancy, is a gene-linked, neurological birth disorder in which the brain degenerates into spongy tissue riddled with microscopic fluid-filled spaces. Canavan disease has been classified as one of a group of genetic disorders known as the leukodystrophies but, unlike most leukodystrophies, both grey and white matter are severely affected in infants with Canavan disease. Recent research has indicated that the cells in the brain responsible for making myelin sheaths, known as oligodendrocytes, cannot properly complete this critical developmental task. Myelin sheaths are the fatty covering that act as insulators around nerve fibers in the brain, as well as providing nutritional support for nerve cells. In Canavan disease, many oligodendrocytes do not mature and instead die, leaving nerve cell projections known as axons vulnerable and unable to properly function. Canavan disease is caused by mutation in the gene for an enzyme called aspartoacylase, which acts to break down the concentrated brain chemical known as N-acetyl-aspartate.

Symptoms of Canavan disease usually appear in the first 3 to 6 months of life and progress rapidly. Symptoms include lack of motor development, feeding difficulties, abnormal muscle tone (weakness or stiffness), and an abnormally large, poorly controlled head. Paralysis, blindness, or hearing loss may also occur. Children are characteristically quiet and apathetic. Although Canavan disease may occur in any ethnic group, it is more frequent among Ashkenazi Jews from eastern Poland, Lithuania, and western Russia, and among Saudi Arabians. Canavan disease can be identified by a simple prenatal blood test that screens for the missing enzyme or for mutations in the gene that controls aspartoacylase. Both parents must be carriers of the defective gene in order to have an affected child. When both parents are found to carry the Canavan gene mutation, there is a one in four (25 percent) chance with each pregnancy that the child will be affected with Canavan disease.

The efficacy of MMF and MMF prodrugs for treating Canavan disease can be determined using animal models and in clinical trials. Suitable animal models for Canavan disease are disclosed for example in Madhavarao, C. N., et al. (2009) J Inherit Metab Dis 32(5), 640-50.

Central Nervous System Vasculitis

Central nervous system vasculitis is an inflammation of, in and around blood vessels, which includes the veins, arteries, and capillaries, and secondary narrowing or blockage of the blood vessels that nourish the brain and spinal cord. Researchers think that inflammation occurs with infection or is thought to be due to a faulty immune system response.

A central nervous system vasculitic syndrome may begin suddenly or develop over time. Symptoms include: headaches, especially a headache that doesn't go away; fever; feeling out-of-sorts; rapid weight loss; confusion or forgetfulness leading to dementia; aches and pains in the joints and muscles; pain while chewing or swallowing; paralysis or numbness, usually in the arms or legs; and visual disturbances, such as double vision, blurred vision, or blindness.

The efficacy of MMF and MMF prodrugs for treating central nervous system vasculitis can be determined using animal models and in clinical trials. Suitable animal models for central nervous system vasculitis are disclosed for example in Malipiero, U., et al. (2006) Brain 129(9), 2404-15.

Charcott-Marie-Tooth Disease

Charcot-Marie-Tooth disease is a muscular atrophy of variable inheritance, beginning in the muscles supplied by the peroneal nerves and progressing slowly to involve the muscles of the hands and arms. The disease is also called Charcot-Marie atrophy or syndrome, peroneal or peroneal muscular atrophy, Marie-Tooth disease, and Tooth's disease. Charcot-Marie-Tooth disease is one of the most common inherited neurological disorders, affecting approximately 1 in 2,500 people in the United States. The disease is named for the three physicians who first identified it in 1886-Jean-Martin Charcot and Pierre Marie in Paris, France, and Howard Henry Tooth in Cambridge, England. Charcot-Marie-Tooth disease, also known as hereditary motor and sensory neuropathy or peroneal muscular atrophy, comprises a group of disorders that affect peripheral nerves. The peripheral nerves lie outside the brain and spinal cord and supply the muscles and sensory organs in the limbs. Disorders that affect the peripheral nerves are called peripheral neuropathies.

The efficacy of MMF and MMF prodrugs for treating Charcott-Marie-Tooth Disease can be determined using animal models and in clinical trials. Suitable animal models for Charcott-Marie-Tooth Disease are disclosed for example in Fledrich, R., et al. (2012) Br Med Bull 102(1), 89-113.

Childhood Ataxia with Central Nervous System Hypomyelination

Childhood ataxia with central nervous system hypomyelination is characterized by ataxia, spasticity, and variable optic atrophy. The phenotypic range includes a prenatal/congenital form, a subacute infantile form (onset age <1 year), an early childhood onset form (onset age 1-5 years), a late childhood/juvenile onset form (onset age 5-15 years), and an adult onset form. The prenatal/congenital form is characterized by severe encephalopathy. In the later onset forms initial motor and intellectual development is normal or mildly delayed followed by neurologic deterioration with a chronic progressive or subacute course. Chronic progressive decline can be exacerbated by rapid deterioration during febrile illnesses or following head trauma or major surgical procedures, or by acute psychological stresses such as extreme fright.

The efficacy of MMF and MMF prodrugs for treating childhood ataxia with central nervous system hypomyelination can be determined using animal models and in clinical trials. Suitable animal models for childhood ataxia with central nervous system hypomyelination are disclosed for example in Geva, M., et al. (2010) Brain 133(8), 2448-61.

Chronic Inflammatory Demyelinating Polyneuropathy

Chronic inflammatory demyelinating polyneuropathy (CIDP) is a neurological disorder characterized by progressive weakness and impaired sensory function in the legs and arms. The disorder, which is sometimes called chronic relapsing polyneuropathy, is caused by damage to the myelin sheath (the fatty covering that wraps around and protects nerve fibers) of the peripheral nerves. Although it can occur at any age and in both genders, CIDP is more common in young adults, and in men more so than women. It often presents with symptoms that include tingling or numbness (beginning in the toes and fingers), weakness of the arms and legs, loss of deep tendon reflexes (areflexia), fatigue, and abnormal sensations. CIDP is closely related to Guillain-Barre syndrome and it is considered the chronic counterpart of that acute disease.

The efficacy of MMF and MMF prodrugs for treating CIDP can be determined using animal models and in clinical trials. Suitable animal models for CIDP are disclosed for example in Ubogu E. E., et al. (2012) Journal of the Peripheral Nervous System 17(1), 53-61; Sajic M. et al. (2012) PLoS ONE 7(2):Article Number: e30708; and Meyer Zu Horste G, et al. (2011) Journal of the Peripheral Nervous System 16 (SUPPL. 3), S87.

Cutaneous Lupus Erythematosus

Cutaneous lupus erythematosus (CLE) can be divided into three main subtypes: acute, subacute, and chronic, all of which demonstrate photosensitivity. Acute cutaneous lupus erythematosus (ACLE) most commonly presents as symmetric erythema overlying the malar cheeks and nasal bridge with sparing of the nasolabial folds (butterfly rash). However, it can also present as a diffuse morbilliform eruption with erythema and edema of the hands, with prominent sparing of the joints. Subacute cutaneous lupus erythematosus (SCLE) characteristically presents as annular or psoriasiform plaques in a photodistribution. Chronic cutaneous lupus erythematosus (CCLE) can be further divided into 3 main types: discoid lupus erythematosus (DLE), tumid lupus, and lupus panniculitis. Tumid lupus typically presents with juicy papules and plaques that heal without scarring, whereas lupus panniculitis involves the subcutaneous tissue, leading to painful subcutaneous nodules that heal with depression and atrophy.

Fumaderm® (dimethyl fumarate and ethyl hydrogen fumarate) has previously been used to treat CLE. See for example Klein, A., et al. (2011), J Eur Acad Dermatol Venereol doi: 10.1111/j.1468-3083.2011.04303.x. The efficacy of MMF and MMF prodrugs for treating CLE can be determined using animal models and in clinical trials. Suitable animal models for CLE are disclosed for example in Furukawa, F. (1997), Lupus 6(2): 193-202.

Chronic Lymphocytic Leukemia

Chronic lymphocytic leukemia, also known as chronic lymphoid leukemia (CLL), is the most common type of adulthood leukemias. Leukemias are cancers of the white blood cells (leukocytes). CLL affects B cell lymphocytes. B cells originate in the bone marrow, develop in the lymph nodes, and normally fight infection by producing antibodies. In CLL, B cells grow out of control and accumulate in the bone marrow and blood, where they crowd out healthy blood cells. CLL is a stage of small lymphocytic lymphoma, a type of B-cell lymphoma, which presents primarily in the lymph nodes. Both are considered the same underlying disease, just with different appearances. CLL is a disease of adults, but, in rare cases, it can occur in teenagers and occasionally in children (inherited). Most (>75%) people newly diagnosed with CLL are over the age of 50, and the majority are men.

The efficacy of MMF and MMF prodrugs for treating chronic lymphocytic leukemia can be determined using animal models and in clinical trials.

### Diabetic Retinopathy

Diabetic retinopathy (also sometimes referred to as diabetic retinitis) is retinopathy (damage to the retina) caused by complications of diabetes, which can eventually lead to blindness. A less serious type is called background retinopathy; a type that often progresses to blindness is called progressive retinopathy. Diabetic retinopathy is the most common diabetic eye disease and a leading cause of blindness in American adults. It is caused by changes in the blood vessels of the retina. In some people with diabetic retinopathy, blood vessels may swell and leak fluid. In other people, abnormal new blood vessels grow on the surface of the retina. Diabetic retinopathy usually affects both eyes.

The efficacy of MMF and MMF prodrugs for treating diabetic retinopathy can be determined using animal models and in clinical trials. Suitable animal models for diabetic retinopathy are disclosed for example in Robinson R., et al. (2012), Disease Models & Mechanisms 5(4):444-56.

Globoid Cell Leukodystrophy

Globoid cell leukodystrophy (also sometimes referred to as Krabbe Disease, galactosylceramide lipidosis, globoid cell leukodystrophy or Krabbe's leukodystrophy) is an inherited metabolic lysosomal storage disease that affects the muscles, vision and mental abilities. It is life-threatening. It begins in infancy with irritability, fretfulness, and rigidity, followed by tonic seizures, convulsions, quadriplegia, blindness, deafness, dysphagia, and progressive mental deterioration. Pathologically, there is rapidly progressive cerebral demyelination and large globoid bodies in the white substance. In people with globoid cell leukodystrophy, the gene mutation affects an enzyme called galactocerebrosidase. Lack of this enzyme causes the buildup of a substance that damages cells that make myelin. This results in damage to the central nervous system. A person gets the disorder when he or she inherits a gene with the mutation from both parents. The disorder can appear soon after birth (early-onset globoid cell leukodystrophy) or in older children or adults (late-onset globoid cell leukodystrophy). The disorder is rare; about 40 cases of globoid cell leukodystrophy are diagnosed in the United States each year.

The efficacy of MMF and MMF prodrugs for treating globoid cell leukodystrophy can be determined using animal models and in clinical trials. Suitable animal models for globoid cell leukodystrophy are disclosed for example in Gentner, B., et al. (2010) Science Translational Medicine 2(58); and Pellegatta, S., et al. (2006) Neurobiology of disease 21(2), 314-23.

Graft Versus Host Disease

Graft versus host disease (also sometimes referred to as graft versus host reaction) is a common complication following an allogeneic tissue transplant. It is commonly associated with stem cell or bone marrow transplant but the term also applies to other forms of tissue graft. Immune cells (white blood cells) in the tissue (the graft) recognize the recipient (the host) as “foreign”. The transplanted immune cells then attack the host's body cells. Graft versus host disease can also occur after a blood transfusion if the blood products used have not been irradiated.

The efficacy of MMF and MMF prodrugs for treating graft versus host disease can be determined using animal models and in clinical trials. Suitable animal models for graft versus host disease are disclosed for example in Noth, R., et al. (2013) American Journal of Physiology—Gastrointestinal and Liver Physiology, 304:7 (G646-G654); and Ka, S. M. (2006) Nephrology Dialysis Transplantation, 21:2 (288-298).

Hepatitis C Viral Infection

Hepatitis C (also sometimes referred to as HCV) is a viral disease of the liver caused by the hepatitis C virus, the most common form of post-transfusion hepatitis; it also follows parenteral drug abuse. It can also spread through sex with an infected person and from mother to baby during childbirth. Hepatitis C viral infection is a common acute sporadic hepatitis, with approximately 50% of acutely infected persons developing chronic hepatitis. Chronic infection is generally mild and asymptomatic, but cirrhosis or hepatocellular cancer may occur.

Most people who are infected with hepatitis C don't have any symptoms for years. A blood test can reveal the presence of the disease. Usually, hepatitis C does not get better by itself. The infection can last a lifetime and may lead to scarring of the liver or liver cancer. Medicines sometimes help, but side effects can be a problem. Serious cases sometimes require a liver transplant.

The efficacy of MMF and MMF prodrugs for treating hepatitis C viral infection can be determined using animal models and in clinical trials. Suitable animal models for hepatitis C are disclosed for example in Meuleman, P., et al. (2011) Antimicrobial Agents and Chemotherapy 55(11), 5159-67; Moriishi, K., et al. (2007) Advanced Drug Delivery Reviews 59(12), 1213-21; and Pan, Q., et al. (2012) Hepatology 55(6), 1673-83.

Herpes Simplex Viral Infection

Herpes simplex is a group of acute infections caused by human herpesviruses 1 and 2, characterized by small fluid-filled vesicles on the skin or a mucous membrane with a raised erythematous base; it may be a primary infection or recurrent because of reactivation of a latent infection. Type 1 herpesvirus infections usually involve nongenital regions of the body, whereas type 2 infections are primarily on the genitals and surrounding areas, although there is overlap between the two types. Precipitating factors include fever, exposure to cold temperature or ultraviolet rays, sunburn, cutaneous or mucosal abrasions, emotional stress, and nerve injury. Oral herpes causes cold sores around the mouth or face. Genital herpes is a sexually transmitted disease (STD). It affects the genitals, buttocks or anal area. Other herpes infections can affect the eyes, skin, or other parts of the body. The virus can be dangerous in newborn babies or in people with weak immune systems.

Dimethyl fumarate has previously been administered to animals infected with the herpes simplex virus and improved the animals' herpes stromal keratitis. See for example Heiligenhaus, A., et al. (2005), Clinical and Experimental Immunology 142(1): 180-187; and Heiligenhaus, A., et al. (2004), Graefe's Archive for Clinical and Experimental Ophthalmology 242(10): 870-877. The efficacy of MMF and MMF prodrugs for treating herpes simplex viral infection can be determined using animal models and in clinical trials. Suitable animal models for herpes simplex are disclosed for example in Huang, W. Y., et al. (2010) Journal of General Virology 91(3), 591-98; and Prichard, M. N., et al. (2011) Antimicrobial Agents and Chemotherapy 55(10), 4728-34.

Human Immunodeficiency Viral Infection

Human immunodeficiency virus (also sometimes referred to as HIV) is a virus of the genus Lentivirus, separable into two serotypes (HIV-1 and HIV-2), that is the etiologic agent of the acquired immunodeficiency syndrome (AIDS), the most advanced stage of infection with HIV. HIV-1, which comprises at least three subgroups (M, N, and O), is of worldwide distribution, while HIV-2 is largely confined to West Africa; transmission and manifestations are similar. The virus kills or damages the body's immune system cells. Transmission is commonly through unprotected sex with an infected person, by sharing drug needles or through contact with the blood of an infected person. Women can pass an HIV infection to their babies during pregnancy or childbirth.

Dimethyl fumarate and monomethyl fumarate have previously been suggested as a neuroprotectant in HIV patients. See for example Cross, S. A., et al. (2011), Journal of Immunology 187(10): 5015-5025. The efficacy of MMF and MMF prodrugs for treating human immunodeficiency viral infection, and/or for use as a neuroprotectant in HIV patients, can be determined using animal models and in clinical trials. Suitable animal models for HIV are disclosed for example in Evans, D. T., et al. (2013), Curr Opin HIV AIDS doi:10.1097/COH.0b013e328361cee8.

Lichen Planus

Lichen planus (also sometimes referred to as lichen ruber planus) is a chronic mucocutaneous disease that affects the skin, tongue, nails and oral mucosa. The disease presents itself in the form of papules, lesions, or rashes. The name lichen planus refers to the dry and undulating, “lichen-like” appearance of affected skin. It is sometimes associated with oxidative stress as well as certain medications and diseases, however the underlying pathology is currently unknown. Lichen planus is characterized by an eruption of violet umbilicated, flat-topped, scaly papules with white lines or puncta (Wickham's striae), which may either be discrete or coalesce to form plaques or other shapes. Lichen planus has many types, including vesicular, hypertrophic, atrophic, follicular, erosive and ulcerative, actinic, and erythematous; most resolve spontaneously, leaving residual hyperpigmentation and atrophy. Lichen planus-like lesions may also be caused by drugs or chemical substances.

A typical lichen planus rash displays with what clinicians call the “6 P's”: well-defined pruritic, planar, purple, polygonal papules and plaques. The commonly affected sites are near the wrist and the ankle. The rash tends to heal with prominent blue-black or brownish discoloration that persists for a long time. Besides the typical lesions, many morphological varieties of the rash may occur. The presence of cutaneous lesions is not constant and may wax and wane over time. Oral lesions tend to last far longer than cutaneous lichen planus lesions.

Fumaderm® (dimethyl fumarate and ethyl hydrogen fumarate) and dimethyl fumarate alone have previously been used to treat lichen planus. See for example Guenther, C. H., et al. (2003), Annals of Pharmacotherapy 37(2): 234-236; and Klein, A., et al. (2012), Journal of the European Academy of Dermatology and Venereology 26(11): 1400-1406. The efficacy of MMF and MMF prodrugs for treating lichen planus can be determined using animal models and in clinical trials. A potential animal model for lichen planus is disclosed in Shi, G., et al. (2010) PLoS ONE 5:10 Article Number: e13216.

Macular Degeneration

Macular degeneration is a medical condition which usually affects older adults and results in a loss of vision in the center of the visual field (the macula) because of damage to the retina. It occurs in “dry” and “wet” forms. It is a major cause of blindness and visual impairment in older adults (>50 years). Macular degeneration can make it difficult or impossible to read or recognize faces, although enough peripheral vision remains to allow other activities of daily life.

Starting from the inside of the eye and going towards the back, the three main layers at the back of the eye are the retina, which contains the nerves; the choroid, which contains the blood supply; and the sclera, which is the white of the eye. The macula is the central area of the retina, which provides the most detailed central vision.

In the dry (nonexudative) form, cellular debris called drusen accumulates between the retina and the choroid, and the retina can become detached. In the wet (exudative) form, which is more severe, blood vessels grow up from the choroid behind the retina, and the retina can also become detached. It can be treated with laser coagulation, and with medication that stops and sometimes reverses the growth of blood vessels. Although some macular dystrophies affecting younger individuals are sometimes referred to as macular degeneration, the term generally refers to age-related macular degeneration.

Age-related macular degeneration begins with characteristic yellow deposits (drusen) in the macula, between the retinal pigment epithelium and the underlying choroid. Most people with these early changes (referred to as age-related maculopathy) have good vision. People with drusen can go on to develop advanced age-related macular degeneration (AMD). The risk is higher when the drusen are large and numerous and associated with disturbance in the pigmented cell layer under the macula. Large and soft drusen are related to elevated cholesterol deposits and may respond to cholesterol-lowering agents.

Based on in vitro testing on human retinal pigment epithelium cells, dimethyl fumarate has been suggested for use in treating macular degeneration. See for example Nelson, K. C., et al. (1999), Investigative Ophthalmology and Visual Science 40(9): 1927-1935; and Winkler, B. S., et al. (1999), Molecular vision 5: 32. The efficacy of MMF and MMF prodrugs for treating macular degeneration can be determined using animal models and in clinical trials. Suitable animal models for macular degeneration are disclosed for example in Catchpole, I., et al. (2013) PLoS ONE 8:6 Article Number: e65518; and Cruz-Guilloty, F., et al. (2013) International Journal of Inflammation Article Number: 503725.

Monomelic Amyotrophy

Monomelic amyotrophy is characterized by progressive degeneration and loss of motor neurons, the nerve cells in the brain and spinal cord that are responsible for controlling voluntary muscles. It is characterized by weakness and wasting in a single limb, usually an arm and hand rather than a foot and leg. There is no pain associated with the disease. Monomelic amyotrophy occurs in males between the ages of 15 and 25. Onset and progression are slow. The disease is seen most frequently in Asia, particularly in Japan and India; it is much less common in North America. In most cases, the cause is unknown, although there have been a few published reports linking monomelic amyotrophy to traumatic or radiation injury. There are also familial forms of monomelic amyotrophy. Diagnosis is made by physical exam and medical history. Electromyography, a special recording technique that detects electrical activity in muscles, shows a loss of the nerve supply, or denervation, in the affected limb; MRI and CT scans may show muscle atrophy.

The efficacy of MMF and MMF prodrugs for treating monomelic amyotrophy can be determined using animal models and in clinical trials.

Necrobiosis Lipoidosis

Necrobiosis lipoidosis, also sometimes called necrobiosis lipoidica, is a degenerative disease of dermal connective tissue characterized by development of erythematous papules or nodules in the pretibial area and sometimes elsewhere, extending to form shiny yellow to red plaques that are covered with telangiectatic vessels and have a scaly, atrophic, depressed center. More than half of affected patients have diabetes; the clinical appearance, genetic background for diabetes, and histopathologic findings are similar in both diabetic and nondiabetic patients.

Fumaderm® (dimethyl fumarate and ethyl hydrogen fumarate) has previously been used to treat necrobiosis lipoidosis. See for example Eberle, F. C., et al. (2010), Acta Derm Venereol 90(1): 104-106; Gambichler, T., et al. (2003), Dermatology 207(4): 422-424; Kreuter, A., et al. (2005), British Journal of Dermatology 153(4): 802-807; and Wang, W. P., et al. (2007), Chinese Journal of Evidence-Based Medicine 7(11): 830-835. The efficacy of MMF and MMF prodrugs for treating necrobiosis lipoidosis can be determined using animal models and in clinical trials.

Neurodegeneration with Brain Iron Accumulation

Neurodegeneration with brain iron accumulation is a rare, inherited, neurological movement disorder characterized by an abnormal accumulation of iron in the brain and progressive degeneration of the nervous system. Symptoms, which vary greatly among patients and usually develop during childhood, may include slow writhing, distorting muscle contractions of the limbs, face, or trunk, choreoathetosis (involuntary, purposeless jerky muscle movements), muscle rigidity (uncontrolled tightness of the muscles), spasticity (sudden, involuntary muscle spasms), ataxia (inability to coordinate movements), confusion, disorientation, seizures, stupor, and dementia. Other less common symptoms may include painful muscle spasms, dysphasia (difficulty speaking), mental retardation, facial grimacing, dysarthria (poorly articulated speech), and visual impairment. Several genes have been found that cause the disease.

The efficacy of MMF and MMF prodrugs for treating neurodegeneration with brain iron accumulation can be determined using animal models and in clinical trials.

Neuromyelitis Optica

Neuromyelitis optica (also referred to as Devic disease, optic neuroencephalomyelopathy, neuro-optic myelitis, and ophthalmoneuromyelitis) is an uncommon disease syndrome of the central nervous system that affects the optic nerves and spinal cord. The disease is marked by diminution of vision and possibly blindness, flaccid paralysis of the extremities, and sensory and genitourinary disturbances. Individuals with the disease develop optic neuritis, which causes pain in the eye and vision loss, and transverse myelitis, which causes weakness, numbness, and sometimes paralysis of the arms and legs, along with sensory disturbances and loss of bladder and bowel control. Neuromyelitis optica leads to loss of myelin, a fatty substance that surrounds nerve fibers and helps nerve signals move from cell to cell. The syndrome can also damage nerve fibers and leave areas of broken-down tissue. In the disease process of neuromyelitis optica, immune system cells and antibodies attack and destroy myelin cells in the optic nerves and the spinal cord.

The efficacy of MMF and MMF prodrugs for treating neuromyelitis optica can be determined using animal models and in clinical trials. Suitable animal models for neuromyelitis optica are disclosed for example in Saadoun, S., et al. (2012) Annals of Neurology 71(3), 323-33; and Tradtrantip, L., et al. (2012) Annals of Neurology 71(3), 314-22.

Neurosarcoidosis

Neurosarcoidosis is a manifestation of sarcoidosis in the nervous system.

Sarcoidosis is a chronic inflammatory disorder that typically occurs in adults between 20 and 40 years of age and primarily affects the lungs, but can also impact almost every other organ and system in the body. Neurosarcoidosis is characterized by inflammation and abnormal cell deposits in any part of the nervous system; the brain, spinal cord, or peripheral nerves. It most commonly occurs in the cranial and facial nerves, the hypothalamus (a specific area of the brain), and the pituitary gland. It is estimated to develop in 5 to 15 percent of those individuals who have sarcoidosis. Weakness of the facial muscles on one side of the face (Bell's palsy) is a common symptom of neurosarcoidosis. The optic and auditory nerves can also become involved, causing vision and hearing impairments. It can cause headache, seizures, memory loss, hallucinations, irritability, agitation, and changes in mood and behavior. Neurosarcoidosis can appear in an acute, explosive fashion or start as a slow chronic illness.

The efficacy of MMF and MMF prodrugs for treating neurosarcoidosis can be determined using animal models and in clinical trials.

Optic Neuritis

Optic neuritis is inflammation or demyelination of the optic nerve, the nerve that transmits light and visual images from the retina to the brain. Because the nerve is located behind (“retro”) the globe of the eye, the condition is also known as retrobulbar neuritis. Optic neuritis is generally experienced as an acute blurring, graying (change in color saturation), or loss of vision, most often in only one eye. It is rare that both eyes are affected at the same time. There may or may not be pain in the affected eye. The pain, when it occurs, can be of several types; dull and aching, pressure-like, or sharp and piercing.

The efficacy of MMF and MMF prodrugs for treating optic neuritis can be determined using animal models and in clinical trials. Suitable animal models for optic neuritis are disclosed for example in Chaudhary, P., et al. (2011) Journal of neuroimmunology 233(1-2), 90-96; and Zhang, J., et al. (2011) International Journal of Ophthalmology 11(1), 43-45.

Pareneoplastic Syndromes

Paraneoplastic syndromes are rare disorders that are triggered by an altered immune system response to a neoplasm. They are defined as clinical syndromes involving nonmetastatic systemic effects that accompany malignant disease. In a broad sense, these syndromes are collections of symptoms that result from substances produced by the tumor, and they occur remotely from the tumor itself. The disease is a symptom-complex arising in a cancer-bearing patient that cannot be explained by local or distant spread of the tumor. The symptoms may be endocrine, neuromuscular or musculoskeletal, cardiovascular, cutaneous, hematologic, gastrointestinal, renal, or miscellaneous in nature.

The efficacy of MMF and MMF prodrugs for treating pareneoplastic syndromes can be determined using animal models and in clinical trials. Suitable animal models for pareneoplastic syndromes are disclosed for example in Barton, B. E., et al. (2000) Proceedings of the Society for Experimental Biology and Medicine 223(2), 190-97; and Diament, M. J., et al. (2006) Cancer Investigation 24(2), 126-31.

Pelizaeus-Merzbacher Disease

Pelizaeus-Merzbacher disease (also referred to as Merzbacher-Pelizaeus disease, familial centrolobar sclerosis, and Pelizaeus-Merzbacher sclerosis) is a rare, progressive, degenerative central nervous system disorder in which coordination, motor abilities, and intellectual function deteriorate. The disease is one of a group of gene-linked disorders known as the leukodystrophies, which affect growth of the myelin sheath, the fatty covering that wraps around and protects nerve fibers in the brain. The disease is caused by a mutation in the gene that controls the production of a myelin protein called proteolipid protein-1 (PLP1). Pelizaeus-Merzbacher disease is inherited as an X-linked recessive trait; the affected individuals are male and the mothers are carriers of the PLP1 mutation. Severity and onset of the disease ranges widely, depending on the type of PLP1 mutation. Pelizaeus-Merzbacher disease is one of a spectrum of diseases associated with PLP1, which also includes Spastic Paraplegia Type 2 (SPG2). The PLP1-related disorders span a continuum of neurologic symptoms that range from severe central nervous system involvement to progressive weakness and stiffness of the legs (SPG2). The disease occurs in early life and runs a slowly progressive course into adolescence or adulthood. It is marked by nystagmus, ataxia, tremor, choreoathetoid movements, parkinsonian facies, dysarthria, and mental deterioration. Pathologically, there is diffuse demyelination in the white substance of the brain, which may involve the brain stem, cerebellum, and spinal cord.

The efficacy of MMF and MMF prodrugs for treating Pelizaeus-Merzbacher disease can be determined using animal models and in clinical trials. Suitable animal models for Pelizaeus-Merzbacher disease are disclosed for example in Wood, P. L., et al. (2011) Lipids in Health and Disease 10; and Yu, L. H., et al. (2012) Molecular Genetics and Metabolism 106(1), 108-14.

Primary Lateral Sclerosis

Primary lateral sclerosis (also sometimes referred to as Erb's syndrome) is a rare neuromuscular disease with slowly progressive weakness in voluntary muscle movement. Primary lateral sclerosis belongs to a group of disorders known as motor neuron diseases. Primary lateral sclerosis affects the upper motor neurons (also called corticospinal neurons) in the arms, legs, and face. It occurs when nerve cells in the motor regions of the cerebral cortex (the thin layer of cells covering the brain which is responsible for most higher level mental functions) gradually degenerate, causing movements to be slow and effortful. Symptoms include weakness, muscle stiffness and spasticity, clumsiness, slowing of movement, and problems with balance and speech. Primary lateral sclerosis is more common in men than in women, with a varied gradual onset that generally occurs between ages 40 and 60. Primary lateral sclerosis progresses gradually over a number of years, or even decades. Scientists do not believe Primary lateral sclerosis has a simple hereditary cause.

The efficacy of MMF and MMF prodrugs for treating primary lateral sclerosis can be determined using animal models and in clinical trials.

Progressive Supranuclear Palsy

Progressive supranuclear palsy (also referred to as Steele-Richardson-Olszewski syndrome) is a rare brain disorder, having onset during the sixth decade, that causes serious and progressive problems with control of gait and balance, along with complex eye movement and thinking problems. The disease is characterized by supranuclear ophthalmoplegia, especially paralysis of the downward gaze, pseudobulbar palsy, dysarthria, dystonic rigidity of the neck and trunk, and dementia. One of the classic signs of the disease is an inability to aim the eyes properly, which occurs because of lesions in the area of the brain that coordinates eye movements. Some individuals describe this effect as a blurring. Affected individuals often show alterations of mood and behavior, including depression and apathy as well as progressive mild dementia.

The efficacy of MMF and MMF prodrugs for treating progressive supranuclear palsy can be determined using animal models and in clinical trials. Suitable animal models for progressive supranuclear palsy are disclosed for example in Lewis, J., et al. (2000) Nature genetics 25(4), 402-05; and O'Leary, J. C., et al. (2010) Molecular Neurodegeneration 5(1).

Schilder's Disease

Schilder's disease (also referred to as encephalitis periaxialis diffusa, Flatau-Schilder disease and Schilder's encephalitis) is a rare progressive demyelinating disorder which usually begins in childhood. The disease is a subacute or chronic form of leukoencephalopathy of children and adolescents. Clinical symptoms include blindness, deafness, bilateral spasticity, aphasia, seizures, personality changes, poor attention, tremors, balance instability, incontinence, muscle weakness, headache, vomiting, speech impairment, progressive mental deterioration and dementia. There is massive destruction of the white substance of the cerebral hemispheres, cavity formation, and glial scarring.

The efficacy of MMF and MMF prodrugs for treating Schilder's Disease can be determined using animal models and in clinical trials.

Subacute Necrotizing Myelopathy

Subacute necrotizing myelopathy (also sometimes referred to as subacute necrotizing encephalopathy or Leigh Disease) is a rare inherited neurometabolic disorder that affects the central nervous system. Subacute necrotizing myelopathy occurs in two forms: the infantile form, which may be the same as pyruvate carboxylase deficiency, is characterized by degeneration of gray matter with necrosis and capillary proliferation in the brain stem; hypotonia, seizures, and dementia; anorexia and vomiting; slow or arrested development; and ocular and respiratory disorders. The disease can be caused by mutations in mitochondrial DNA or by deficiencies of an enzyme called pyruvate dehydrogenase. Symptoms of the disease usually progress rapidly. The earliest signs may be poor sucking ability, the loss of head control and motor skills. These symptoms may be accompanied by loss of appetite, vomiting, irritability, continuous crying, and seizures. As the disorder progresses, symptoms may also include generalized weakness, lack of muscle tone, and episodes of lactic acidosis, which can lead to impairment of respiratory and kidney function. Death usually occurs before age 3. The adult form usually first manifests as bilateral optic atrophy with central scotoma and colorblindness; then there is a quiescent period of up to 30 years; and then late symptoms appear such as ataxia, spastic paresis, clonic jerks, grand mal seizures, psychic lability, and mild dementia.

The efficacy of MMF and MMF prodrugs for treating subacute necrotizing myelopathy can be determined using animal models and in clinical trials. Suitable animal models for subacute necrotizing myelopathy are disclosed for example in Quintana, A., et al. (2012), Journal of Clinical Investigation 122:7 (2359-2368).

Susac's Syndrome

Susac's syndrome is a rare disorder characterized by impaired brain function (encephalopathy), blockage (occlusion) of the arteries that supply blood to the retina (branched retinal arterial occlusion), and hearing loss. Two main forms of Susac's syndrome have been identified. In one form, encephalopathy is the main finding, in the other form, branched retinal arterial occlusion and hearing loss occur without signs of brain disease. The specific symptoms and severity of Susac's syndrome vary from one person to another. The encephalopathic form of Susac's syndrome often improves spontaneously even without treatment (self-limited); the other form is frequently a chronic disorder. Although considered rare, Susac's syndrome is being recognized more often worldwide and its true frequency in the general population is unknown. Susac's syndrome is considered an autoimmune endotheliopathy.

The efficacy of MMF and MMF prodrugs for treating Susac's syndrome can be determined using animal models and in clinical trials.

Transverse Myelitis

Transverse myelitis is a neurological disorder caused by inflammation across both sides of one level, or segment, of the spinal cord. The term myelitis refers to inflammation of the spinal cord; transverse simply describes the position of the inflammation, that is, across the width of the spinal cord. Attacks of inflammation can damage or destroy myelin, the fatty insulating substance that covers nerve cell fibers. This damage causes nervous system scars that interrupt communications between the nerves in the spinal cord and the rest of the body. Symptoms of transverse myelitis include a loss of spinal cord function over several hours to several weeks.

The efficacy of MMF and MMF prodrugs for treating transverse myelitis can be determined using animal models and in clinical trials.

Tumors

The term “tumor” is a commonly used synonym for a neoplasm, a solid or fluid-filled lesion, that may or may not be cystic, and may or may not be formed by an abnormal growth of neoplastic cells. Tumors typically appear as enlarged lesions. The term “tumor” is not synonymous with cancer, because it is not necessarily malignant. A tumor can be any one of benign, pre-malignant, or malignant and can also represent as a lesion without cancerous potential.

A tumor is typically caused by an abnormal proliferation of tissues, which may or may not be caused by one or more genetic mutations. Not all tumors cause a tumorous overgrowth of tissue, however.

In certain embodiments, a tumor is a solid tumor.

In certain embodiments, a the solid tumor is one of mamma carcinoma, colon carcinoma, melanoma, primary liver cell carcinoma, adenocarcinoma, kaposi's sarcoma, prostate carcinoma, multiple myeloma (plasmocytoma), Burkitt lymphoma, and Castleman tumor.

The efficacy of MMF and MMF prodrugs for treating tumors can be determined using animal models and in clinical trials.

Zellweger Syndrome

Zellweger syndrome (also referred to as cerebrohepatorenal syndrome) is an autosomal recessive disorder characterized by craniofacial abnormalities, hypotonia, hepatomegaly, polycystic kidneys, jaundice, and death in early infancy, and associated with absence of peroxisomes in the liver and kidneys. The disease is one of a group of four related diseases called peroxisome biogenesis disorders, which are part of a larger group of diseases known as the leukodystrophies. These are inherited conditions that damage the white matter of the brain and also affect how the body metabolizes particular substances in the blood and organ tissues. The diseases are caused by defects in any one of 13 genes, termed PEX genes, required for the normal formation and function of peroxisomes. The disorders are divided into two groups: Zellweger spectrum disorders and Rhizomelic Chondrodysplasia Punctua spectrum disorders. The Zellweger spectrum is comprised of three disorders that have considerable overlap of features. These include Zellweger syndrome (the most severe form), neonatal adrenoleukodystrophy, and Infantile Refsum disease (the least severe form).

The efficacy of MMF and MMF prodrugs for treating Zellweger syndrome can be determined using animal models and in clinical trials. Suitable animal models for Zellweger syndrome are disclosed for example in Mast, F. D., et al. (2011) DMM Disease Models and Mechanisms 4(5), 659-72; and Muller, C. C., et al. (2011) DMM Disease Models and Mechanisms 4(1), 104-19.

### Administration

MMF and/or a prodrug of MMF and pharmaceutical compositions thereof may be administered orally or by any other appropriate route, for example, by infusion or bolus injection, by absorption through epithelial or mucocutaneous linings (e.g., oral mucosa, rectal, and intestinal mucosa, etc.). Other suitable routes of administration include, but are not limited to, intradermal, intramuscular, intraperitoneal, intravenous, subcutaneous, intranasal, epidural, oral, sublingual, intracerebral, intravaginal, transdermal, rectal, inhalation, or topical.

Administration may be systemic or local. Various delivery systems are known, e.g., encapsulation in liposomes, microparticles, microcapsules, capsules, etc., that may be used to administer a compound and/or pharmaceutical composition.

The amount of MMF and/or a prodrug of MMF that will be effective in the treatment of a disease in a patient will depend, in part, on the nature of the condition and can be determined by standard clinical techniques known in the art. In addition, in vitro or in vivo assays may be employed to help identify optimal dosage ranges. A therapeutically effective amount of MMF and/or a prodrug of MMF to be administered may also depend on, among other factors, the subject being treated, the weight of the subject, the severity of the disease, the manner of administration, and the judgment of the prescribing physician. In the case of an MMF prodrug, for which MMF is the pharmacologically active metabolite, the amount of prodrug to be administered is generally determined by calculating the weight of any pharmacologically inactive promoiety that is cleaved during metabolism of the prodrug and then administering a MMF equivalent amount of the prodrug.

For systemic administration, a therapeutically effective dose may be estimated initially from in vitro assays. For example, a dose may be formulated in animal models to achieve a beneficial circulating composition concentration range. Initial doses may also be estimated from in vivo data, e.g., animal models, using techniques that are known in the art. Such information may be used to more accurately determine useful doses in humans. One having ordinary skill in the art may optimize administration to humans based on animal data.

A dose may be administered in a single dosage form or in multiple dosage forms. When multiple dosage forms are used the amount of compound contained within each dosage form may be the same or different. The amount of MMF and/or a prodrug of MMF contained in a dose may depend on the route of administration and whether the disease in a patient is effectively treated by acute, chronic, or a combination of acute and chronic administration.

In certain embodiments an administered dose is less than a toxic dose. Toxicity of the compositions described herein may be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. In certain embodiments, MMF and/or a prodrug of MMF may exhibit a high therapeutic index. The data obtained from these cell culture assays and animal studies may be used in formulating a dosage range that is not toxic for use in humans. A dose of MMF and/or a prodrug of MMF provided by the present disclosure may be within a range of circulating concentrations in for example the blood, plasma, or central nervous system, that include the effective dose and that exhibits little or no toxicity. A dose may vary within this range depending upon the dosage form employed and the route of administration utilized. In certain embodiments, an escalating dose may be administered.

### Combination Therapy

Methods provided by the present disclosure further comprise administering one or more pharmaceutically active compounds in addition to MMF and/or a prodrug of MMF. Such compounds may be provided to treat the same disease or a different disease than the disease being treated with the MMF and/or MMF prodrug.

In certain embodiments, MMF and/or an MMF prodrug may be used in combination with at least one other therapeutic agent. In certain embodiments, MMF and/or a MMF prodrug may be administered to a patient together with another compound for treating diseases and conditions including: adrenal leukodystrophy, Alexanders Disease, Alpers' Disease, balo concentric sclerosis, bronchiolitis obliterans organizing pneumonia, Canavan disease, central nervous system vasculitis, Charcott-Marie-Tooth Disease, childhood ataxia with central nervous system hypomyelination, chronic inflammatory demyelinating polyneuropathy, cutaneous lupus erythematosus, chronic lymphocytic leukemia, diabetic retinopathy, globoid cell leukodystrophy, graft versus host disease, hepatitis C viral infection, herpes simplex viral infection, human immunodeficiency viral infection, lichen planus, macular degeneration, monomelic amyotrophy, necrobiosis lipoidosis, neurodegeneration with brain iron accumulation, neuromyelitis optica, neurosarcoidosis, optic neuritis, pareneoplastic syndromes, Pelizaeus-Merzbacher disease, primary lateral sclerosis, progressive supranuclear palsy, Schilder's Disease, subacute necrotizing myelopathy, Susac syndrome, transverse myelitis, a tumor and Zellweger syndrome.

MMF and/or an MMF prodrug and the at least one other therapeutic agent may act additively or, and in certain embodiments, synergistically. The at least one additional therapeutic agent may be included in the same dosage form as MMF and/or the MMF prodrug or may be provided in a separate dosage form. Methods provided by the present disclosure can further include, in addition to administering MMF and/or an MMF prodrug, administering one or more therapeutic agents effective for treating the same or different disease than the disease being treated by MMF and/or the MMF prodrug. Methods provided by the present disclosure include administration of MMF and/or an MMF prodrug and one or more other therapeutic agents provided that the combined administration does not inhibit the therapeutic efficacy of the MMF and/or the MMF prodrug and/or does not typically produce significant and/or substantial adverse combination effects.

In certain embodiments, dosage forms comprising MMF and/or a prodrug of MMF may be administered concurrently with the administration of another therapeutic agent, which may be part of the same dosage form as, or in a different dosage form than that comprising MMF and/or a prodrug of MMF. MMF and/or a prodrug of MMF may be administered prior or subsequent to administration of another therapeutic agent. In certain embodiments of combination therapy, the combination therapy may comprise alternating between administering MMF and/or a prodrug of MMF and a composition comprising another therapeutic agent, e.g., to minimize adverse drug effects associated with a particular drug. When MMF and/or a prodrug of MMF is administered concurrently with another therapeutic agent that potentially may produce an adverse drug effect including, but not limited to, toxicity, the other therapeutic agent may advantageously be administered at a dose that falls below the threshold at which the adverse drug reaction is elicited.

In certain embodiments, dosage forms comprising MMF and/or a prodrug of MMF may be administered with one or more substances to enhance, modulate and/or control release, bioavailability, therapeutic efficacy, therapeutic potency, stability, and the like of MMF and/or a prodrug of MMF. For example, to enhance the therapeutic efficacy of a MMF and/or a prodrug of MMF, the MMF and/or a prodrug of MMF may be co-administered with or a dosage form comprising MMF and/or a prodrug of MMF may comprise one or more active agents to increase the absorption or diffusion of MMF and/or a prodrug of MMF from the gastrointestinal tract to the systemic circulation, or to inhibit degradation of the MMF and/or a prodrug of MMF in the blood of a patient. In certain embodiments, MMF and/or a prodrug of MMF may be co-administered with an active agent having pharmacological effects that enhance the therapeutic efficacy of a MMF and/or a prodrug of MMF.

In certain embodiments, MMF and/or a MMF prodrug disclosed herein, or a pharmaceutical composition thereof, may be administered to a patient for treating adrenal leukodystrophy in combination with a therapy or another therapeutic agent known or believed to be useful in treating adrenal leukodystrophy, for example a steroid such as cortisol.

In certain embodiments, MMF and/or a MMF prodrug disclosed herein, or a pharmaceutical composition thereof, may be administered to a patient for treating Alexanders Disease in combination with a therapy or another therapeutic agent known or believed to be useful in treating Alexanders Disease, for example an anticonvulsant.

In certain embodiments, MMF and/or a MMF prodrug disclosed herein, or a pharmaceutical composition thereof, may be administered to a patient for treating Alpers' Disease in combination with a therapy or another therapeutic agent known or believed to be useful in treating Alpers' Disease, for example an anticonvulsant.

In certain embodiments, MMF and/or a MMF prodrug disclosed herein, or a pharmaceutical composition thereof, may be administered to a patient for treating balo concentric sclerosis in combination with a therapy or another therapeutic agent known or believed to be useful in treating balo concentric sclerosis, for example a corticosteroid such as methyl prednisolone.

In certain embodiments, MMF and/or a MMF prodrug disclosed herein, or a pharmaceutical composition thereof, may be administered to a patient for treating bronchiolitis obliterans organizing pneumonia in combination with a therapy or another therapeutic agent known or believed to be useful in treating bronchiolitis obliterans organizing pneumonia.

In certain embodiments, MMF and/or a MMF prodrug disclosed herein, or a pharmaceutical composition thereof, may be administered to a patient for treating Canavan disease in combination with a therapy or another therapeutic agent known or believed to be useful in treating Canavan disease.

In certain embodiments, MMF and/or a MMF prodrug disclosed herein, or a pharmaceutical composition thereof, may be administered to a patient for treating central nervous system vasculitis in combination with a therapy or another therapeutic agent known or believed to be useful in treating central nervous system vasculitis, for example a steroid and/or an immunosuppressive drug, such as prednisolone and cyclophosphamide.

In certain embodiments, MMF and/or a MMF prodrug disclosed herein, or a pharmaceutical composition thereof, may be administered to a patient for treating Charcott-Marie-Tooth Disease in combination with a therapy or another therapeutic agent known or believed to be useful in treating Charcott-Marie-Tooth Disease, for example a pain killing drug.

In certain embodiments, MMF and/or a MMF prodrug disclosed herein, or a pharmaceutical composition thereof, may be administered to a patient for treating childhood ataxia with central nervous system hypomyelination in combination with a therapy or another therapeutic agent known or believed to be useful in treating childhood ataxia with central nervous system hypomyelination, for example an antibiotic, ursodeoxycholic acid and/or gabapentin.

In certain embodiments, MMF and/or a MMF prodrug disclosed herein, or a pharmaceutical composition thereof, may be administered to a patient for treating Chronic inflammatory demyelinating polyneuropathy (CIDP) in combination with a therapy or another therapeutic agent known or believed to be useful in treating CIDP, for example a corticosteroid such as predisone and/or intravenous immunoglobulin therapy.

In certain embodiments, MMF and/or a MMF prodrug disclosed herein, or a pharmaceutical composition thereof, may be administered to a patient for treating cutaneous lupus erythematosus in combination with a therapy or another therapeutic agent known or believed to be useful in treating cutaneous lupus erythematosus, for example hydroxychloroquine, chloroquine, quinacrine, corticosteroids, methotrexate, mycophenolate mofetil, azathioprine, topical or intralesional corticosteroids and antimalarials, topical calcineurin inhibitors, topical retinoids, topical imiquimod, methotrexate, mycophenolate mofetil, thalidomide and/or sun-protective measures including use of sunscreens.

In certain embodiments, MMF and/or a MMF prodrug disclosed herein, or a pharmaceutical composition thereof, may be administered to a patient for treating chronic lymphocytic leukemia in combination with a therapy or another therapeutic agent known or believed to be useful in treating chronic lymphocytic leukemia.

In certain embodiments, MMF and/or a MMF prodrug disclosed herein, or a pharmaceutical composition thereof, may be administered to a patient for treating diabetic retinopathy in combination with a therapy or another therapeutic agent known or believed to be useful in treating diabetic retinopathy.

In certain embodiments, MMF and/or a MMF prodrug disclosed herein, or a pharmaceutical composition thereof, may be administered to a patient for treating globoid cell leukodystrophy in combination with a therapy or another therapeutic agent known or believed to be useful in treating globoid cell leukodystrophy, for example an anticonvulsant or an antispasticity drug.

In certain embodiments, MMF and/or a MMF prodrug disclosed herein, or a pharmaceutical composition thereof, may be administered to a patient for treating graft versus host disease in combination with a therapy or another therapeutic agent known or believed to be useful in treating graft versus host disease, for example immune suppression drugs, including steroids, ciclosporin, tacrolimus, anti-lymphocytic or anti-thymocytic globulin, monoclonal antibodies, mycophenolate, mofetil, pentostatin, azathioprine, methotrexate, thalidomide, sirolimus, everolimus, imatinib, clofazamine, halofuginone, etanercept, hydroxychloroquine and etretinate.

In certain embodiments, MMF and/or a MMF prodrug disclosed herein, or a pharmaceutical composition thereof, may be administered to a patient for treating hepatitis C viral infection in combination with a therapy or another therapeutic agent known or believed to be effective in treating hepatitis C viral infection, for example interferons, ribavirin, boceprivir and/or telaprevir.

In certain embodiments, MMF and/or a MMF prodrug disclosed herein, or a pharmaceutical composition thereof, may be administered to a patient for treating herpes simplex viral infection in combination with a therapy or another therapeutic agent known or believed to be useful in treating herpes simplex viral infection, for example acyclovir, valacyclovir, famciclovir and/or penciclovir.

In certain embodiments, MMF and/or a MMF prodrug disclosed herein, or a pharmaceutical composition thereof, may be administered to a patient for treating human immunodeficiency viral infection in combination with a therapy or another therapeutic agent known or believed to be useful in treating human immunodeficiency viral infection, for example non-nucleoside reverse transcriptase inhibitors (NNRTIs) such as efavirenz, etravirine, nevirapine; nucleoside reverse transcriptase inhibitors (NRTIs) such as abacavir, the combination drugs emtricitabine and tenofovir, and lamivudine and zidovudine; protease inhibitors (PIs) such as atazanavir, darunavir, fosamprenavir and ritonavir; entry or fusion inhibitors such as enfuvirtide and maraviroc; and/or integrase inhibitors such as raltegravir.

In certain embodiments, MMF and/or a prodrug of MMF disclosed herein, or a pharmaceutical composition thereof, may be administered to a patient for treating lichen planus in combination with a therapy or another therapeutic agent known or believed to be useful in treating lichen planus, for example corticosteroids, retinoids, nonsteroidal creams or ointments (topical calcineurin inhibitors: tacrolimus and pimecrolimus), antihistamines and/or phototherapy.

In certain embodiments, MMF and/or a prodrug of MMF disclosed herein, or a pharmaceutical composition thereof, may be administered to a patient for treating macular degeneration in combination with a therapy or another therapeutic agent known or believed to be effective in treating macular degeneration, for example vitamin C, vitamin E, beta carotene, vitamin A, zinc/zinc oxide and/or copper/cupric oxide.

In certain embodiments, MMF and/or a prodrug of MMF disclosed herein, or a pharmaceutical composition thereof, may be administered to a patient for treating monomelic amyotrophy in combination with a therapy or another therapeutic agent known or believed to be useful in treating monomelic amyotrophy.

In certain embodiments, MMF and/or a prodrug of MMF disclosed herein, or a pharmaceutical composition thereof, may be administered to a patient for treating necrobiosis lipoidosis in combination with a therapy or another therapeutic agent known or believed to be useful in treating necrobiosis lipoidosis, for example pentoxifylline, aspirin, ticlopidine, nicotinamide, clofazimine, heparin, tretinoin and/or laser treatments.

In certain embodiments, MMF and/or a prodrug of MMF disclosed herein, or a pharmaceutical composition thereof, may be administered to a patient for treating neurodegeneration with brain iron accumulation in combination with a therapy or another therapeutic agent known or believed to be useful in treating neurodegeneration with brain iron accumulation.

In certain embodiments, MMF and/or a prodrug of MMF disclosed herein, or a pharmaceutical composition thereof, may be administered to a patient for treating neuromyelitis optica in combination with a therapy or another therapeutic agent known or believed to be useful in treating neuromyelitis optica, for example corticosteroids, plasma exchange, immunosuppressive medications such as azathioprine, mycophenolate mofetil and/or rituximab.

In certain embodiments, MMF and/or a prodrug of MMF disclosed herein, or a pharmaceutical composition thereof, may be administered to a patient for treating neurosarcoidosis in combination with a therapy or another therapeutic agent known or believed to be useful in treating neurosarcoidosis, for example corticosteroids, immunomodulatory drugs such as hydroxychloroquine, pentoxyfilline, thalidomide, and infliximab, and/or immunosuppressive drugs such as methotrexate, azathioprine, cyclosporin, and cyclophosphamide.

In certain embodiments, MMF and/or a prodrug of MMF disclosed herein, or a pharmaceutical composition thereof, may be administered to a patient for treating optic neuritis in combination with a therapy or another therapeutic agent known or believed to be useful in treating optic neuritis, for example corticosteroids, interferon beta-1a and/or interferon beta-1b.

In certain embodiments, MMF and/or a prodrug of MMF disclosed herein, or a pharmaceutical composition thereof, may be administered to a patient for treating pareneoplastic syndromes in combination with a therapy or another therapeutic agent known or believed to be effective in treating pareneoplastic syndromes, for example corticosteroids, immunosuppressants (e.g., azathioprine, cyclophosphamide), anti-seizure medications (e.g., carbamazepine, valproic acid), medications to enhance nerve-to-muscle transmission (e.g., 3,4-diaminopyridine, pyridostigmine), plasmapheresis and/or intravenous immune globulin.

In certain embodiments, MMF and/or a prodrug of MMF disclosed herein, or a pharmaceutical composition thereof, may be administered to a patient for treating Pelizaeus-Merzbacher disease in combination with a therapy or another therapeutic agent known or believed to be useful in treating Pelizaeus-Merzbacher disease, for example antispasticity agents, including intrathecal baclofen, tizanidine and/or benzodiazepines.

In certain embodiments, MMF and/or a prodrug of MMF disclosed herein, or a pharmaceutical composition thereof, may be administered to a patient for treating primary lateral sclerosis in combination with a therapy or another therapeutic agent known or believed to be useful in treating primary lateral sclerosis, for example antispasticity drugs such as baclofen, tizanidine, diazepam, clonazepam; and/or medications to treat cramps or pain related to spasticity, including phenyloin and analgesics.

In certain embodiments, MMF and/or a prodrug of MMF disclosed herein, or a pharmaceutical composition thereof, may be administered to a patient for treating progressive supranuclear palsy in combination with a therapy or another therapeutic agent known or believed to be useful in treating progressive supranuclear palsy, for example carbidopa-levodopa, dopamine agonists, amantadine; antidepressants such as fluoxetine, imipramine and amitriptyline; botulinum toxin (Botox), coenzyme Q-10, lithium, valproic acid and/or davunetide.

In certain embodiments, MMF and/or a prodrug of MMF disclosed herein, or a pharmaceutical composition thereof, may be administered to a patient for treating Schilder's Disease in combination with a therapy or another therapeutic agent known or believed to be useful in treating Schilder's Disease, for example corticosteroids, beta-interferon and/or immunosuppressive drugs.

In certain embodiments, MMF and/or a prodrug of MMF disclosed herein, or a pharmaceutical composition thereof, may be administered to a patient for treating subacute necrotizing myelopathy in combination with a therapy or another therapeutic agent known or believed to be useful in treating subacute necrotizing myelopathy, for example thiamine (vitamin B1), sodium bicarbonate, sodium citrate and/or dichloroacetate.

In certain embodiments, MMF and/or a prodrug of MMF disclosed herein, or a pharmaceutical composition thereof, may be administered to a patient for treating Susac syndrome in combination with a therapy or another therapeutic agent known or believed to be useful in treating Susac syndrome, for example corticosteroids, antiplatelets, anticoagulants and/or cyclophosphamide.

In certain embodiments, MMF and/or a prodrug of MMF disclosed herein, or a pharmaceutical composition thereof, may be administered to a patient for treating transverse myelitis in combination with a therapy or another therapeutic agent known or believed to be useful in treating transverse myelitis, for example intravenous steroids, plasma exchange therapy, pain medications including acetaminophen, ibuprofen and naproxen; antidepressants such as sertraline; anticonvulsants, such as gabapentin and pregabalin; antispasticity drugs such as baclofen; drugs for treating urinary or bowel dysfunction; and or antidepressants.

In certain embodiments, MMF and/or a prodrug of MMF disclosed herein, or a pharmaceutical composition thereof, may be administered to a patient for treating a tumor in combination with a therapy or another therapeutic agent known or believed to be useful in treating a tumor, for example vinblastine, vincristine, methotrexate, uracil, 5-fluorouracil and gemcitabine.

In certain embodiments, MMF and/or a prodrug of MMF disclosed herein, or a pharmaceutical composition thereof, may be administered to a patient for treating Zellweger syndrome in combination with a therapy or another therapeutic agent known or believed to be useful in treating Zellweger syndrome, for example anticonvulsants.

Finally, it should be noted that there are alternative ways of implementing the embodiments disclosed herein. Accordingly, the present embodiments are to be considered as illustrative and not restrictive, and the claims are not to be limited to the details given herein, but may be modified within the scope and equivalents thereof.

1. A method of treating a disease in a patient comprising administering to a patient in need of such treatment a therapeutically effective amount of a compound selected from: (i) monomethyl fumarate, (ii) a prodrug of monomethyl fumarate, and (iii) combinations thereof, wherein the disease is chosen from balo concentric sclerosis, bronchiolitis obliterans organizing pneumonia, central nervous system vasculitis, Charcott-Marie-Tooth Disease, childhood ataxia with central nervous system hypomyelination, diabetic retinopathy, graft versus host disease, monomelic amyotrophy, neurodegeneration with brain iron accumulation, neurosarcoidosis, pareneoplastic syndromes, subacute necrotizing myelopathy, Susac syndrome and transverse myelitis.

2. The method of claim 1, wherein the compound comprises monomethyl fumarate.

3. The method of claim 1, wherein the compound comprises a prodrug of monomethyl fumarate.

4. The method of claim 3, wherein the compound comprises dimethyl fumarate.

5. The method of claim 3, wherein the compound is a compound of Formula (I): 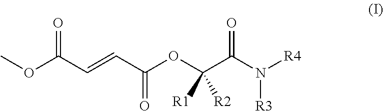
or a pharmaceutically acceptable salt thereof, wherein:
R¹ and R² are independently chosen from hydrogen, C₁₋₆ alkyl, and substituted C₁₋₆ alkyl;
R³ and R⁴ are independently chosen from hydrogen, C₁₋₆ alkyl, substituted C₁₋₆ alkyl, C₁₋₆ heteroalkyl, substituted C₁₋₆ heteroalkyl, C₄₋₁₂ cycloalkylalkyl, substituted C₄₋₁₂ cycloalkylalkyl, C₇₋₁₂ arylalkyl, and substituted C₇₋₁₂ arylalkyl; or R³ and R⁴ together with the nitrogen to which they are bonded form a ring chosen from a C₅₋₁₀ heteroaryl, substituted C₅₋₁₀ heteroaryl, C₅₋₁₀ heterocycloalkyl, and substituted C₅₋₁₀ heterocycloalkyl;
wherein each substituent group is independently chosen from halogen, —OH, —CN, —CF₃, ═O, —NO₂, benzyl, —C(O)NR¹¹₂, —R¹¹, —OR¹¹, —C(O)R¹¹, —COOR¹¹, and —NR¹¹₂ wherein each R¹¹ is independently chosen from hydrogen and C₁₋₄ alkyl.

6. The method of claim 5, wherein each of R¹ and R² is hydrogen.

7. The method of claim 5, wherein one of R¹ and R² is hydrogen and the other of R¹ and R² is chosen from methyl, ethyl, n-propyl, isopropyl, n-butyl, isobutyl, and sec-butyl.

8. The method of claim 5, wherein R³ and R⁴ are independently chosen from hydrogen and C₁₋₆ alkyl.

9. The method of claim 5, wherein R³ and R⁴ together with the nitrogen to which they are bonded form a C₅₋₁₀ heterocycloalkyl ring.

10. The method of claim 5, wherein one of R¹ and R² is hydrogen and the other of R¹ and R² is chosen from hydrogen and C₁₋₆ alkyl; and R³ and R⁴ together with the nitrogen to which they are bonded form a ring chosen from morpholine, piperazine, and N-substituted piperazine.

11. The method of claim 5, wherein one of R¹ and R² is hydrogen; and the other of R¹ and R² is chosen from hydrogen and C₁₋₆ alkyl; R³ is hydrogen; and R⁴ is chosen from hydrogen, C₁₋₆ alkyl, and benzyl.

12. The method of claim 5, wherein the compound is chosen from:
(N,N-diethylcarbamoyl)methyl methyl(2E)but-2-ene-1,4-dioate;
methyl [N-benzylcarbamoyl]methyl (2E)but-2-ene-1,4-dioate;
methyl 2-morpholin-4-yl-2-oxoethyl (2E)but-2-ene-1,4-dioate;
(N-butylcarbamoyl)methyl methyl(2E)but-2-ene-1,4-dioate;
[N-(2-methoxyethyl)carbamoyl]methyl methyl(2E)but-2-ene-1,4-dioate;
methyl(N-(1,3,4-thiadiazol-2yl)carbamoyl)methyl(2E)but-2ene-1,4-dioate;
(N,N-dimethylcarbamoyl)methyl methyl(2E)but-2-ene-1,4-dioate;
(N-methoxy-N-methylcarbamoyl)methyl methyl(2E)but-2-ene-1,4-dioate;
bis-(2-methoxyethylamino)carbamoyl]methyl methyl(2E)but-2-ene-1,4-dioate;
[N-(methoxycarbonyl)carbamoyl]methyl methyl(2E)but-2ene-1,4-dioate;
methyl 2-oxo-2-piperazinylethyl (2E)but-2-ene-1,4-dioate;
methyl 2-oxo-2-(2-oxo(1,3-oxazolidin-3yl)ethyl (2E)but-2ene-1,4-dioate;
{N-[2-(dimethylamino)ethyl]carbamoyl}methyl methyl(2E)but-2ene-1,4 dioate;
methyl 2-(4-methylpiperazinyl)-2-oxoethyl (2E)but-2-ene-1.4-dioate;
methyl {N—[(propylamino)carbonyl]carbamoyl}methyl (2E)but-2ene-1,4-dioate;
2-(4-acetylpiperazinyl)-2-oxoethyl methyl (2E)but-2ene-1,4-dioate;
{N,N-bis[2-(methylethoxy)ethyl]carbamoyl}methyl methyl(2E)but-2-ene-1,4-dioate; methyl 2-(4-benzylpiperazinyl)-2-oxoethyl (2E)but-2-ene-1.4-dioate;
[N,N-bis(2-ethoxyethyl)carbamoyl]methyl methyl(2E)but-2-ene-1,4-dioate;
2-{(2S)-2-[(tert-butyl)oxycarbonyl]pyrrolidinyl}-2-oxoethyl methyl (2E)but-2ene-1,4-dioate;
(N-{[tert-butyl)oxycarbonyl]methyl}-N-methylcarbamoyl)methyl methyl(2E)but-2ene-1,4-dioate;
{N-(ethoxycarbonyl)methyl]-N-methylcarbamoyl}methyl methyl(2E)but-2-ene-1,4-dioate;
methyl 1-methyl-2-morpholin-4-yl-2-oxoethyl (2E)but-2-ene-1,4-dioate;
[N,N-bis(2-methoxyethyl)carbamoyl]ethyl methyl (2E)but-2-ene-1,4-dioate;
(N,N-dimethylcarbamoyl)ethyl methyl (2E)but-2-ene-1,4-dioate;
(N-{[(tert-butyl)oxycarbonyl]methyl}carbamoyl)methyl methyl(2E)but-2-ene-1,4-dioate;
methyl (N-methyl-N-{[(methylethyl)oxycarbonyl]methyl}carbamoyl)methyl (2E)but-2-ene-1,4-dioate;
{N-[(ethoxycarbonyl)methyl]-N-benzylcarbamoyl}methyl methyl(2E)but-2-ene-1,4-dioate;
{N-[(ethoxycarbonyl)methyl]-N-benzylcarbamoyl}ethyl methyl (2E)but-2-ene-1,4-dioate;
{N-[(ethoxycarbonyl)methyl]-N-methylcarbamoyl}ethyl methyl (2E)but-2-ene-1,4-dioate;
(1S)-1-methyl-2-morpholin-4-yl-2-oxoethyl methyl (2E)but-2-ene-1,4-dioate;
(1S)-1-[N,N-bis(2-methoxyethyl)carbamoyl]ethyl methyl (2E)but-2-ene-1,4-dioate;
(1R)-1-(N,N-diethylcarbamoyl)ethyl methyl (2E)but-2-ene-1,4-dioate;
(N-[(methoxycarbonyl)ethyl]carbamoyl)methyl methyl(2E)but-2-ene-1,4-dioate;
and
a pharmaceutically acceptable salt of any of the foregoing.

13. The method of claim 3, wherein the compound is compound of Formula (II): 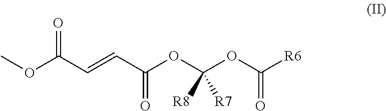
or a pharmaceutically acceptable salt thereof, wherein:
R⁶ is chosen from C₁₋₆ alkyl, substituted C₁₋₆ alkyl, C₁₋₆ heteroalkyl, substituted C₁₋₆ heteroalkyl, C₃₋₈cycloalkyl, substituted C₃₋₈cycloalkyl, C₆₋₈aryl, substituted C₆₋₈aryl, and —OR¹⁰ wherein R¹⁰ is chosen from C₁₋₆ alkyl, substituted C₁₋₆ alkyl, C₃₋₁₀ cycloalkyl, substituted C₃₋₁₀ cycloalkyl, C₆₋₁₀ aryl, and substituted C₆₋₁₀ aryl; and R⁷ and R⁸ are independently chosen from hydrogen, C₁₋₆ alkyl, and substituted C₁₋₆ alkyl;
wherein each substituent group is independently chosen from halogen, —OH, —CN, —CF₃, ═O, —NO₂, benzyl, —C(O)NR¹¹₂, —R¹¹, —OR¹¹, —C(O)R¹¹, —COOR¹¹, and —NR¹¹₂ wherein each R¹¹ is independently chosen from hydrogen and C₁₋₄ alkyl.

14. The method of claim 13, wherein one of R⁷ and R⁸ is hydrogen and the other of R⁷ and R⁸ is chosen from methyl, ethyl, n-propyl, and isopropyl.

15. The method of claim 13, wherein each substituent group is —OR¹¹ wherein each R¹¹ is C₁₋₄alkyl.

16. The method of claim 13, wherein R⁶ is C₁₋₆alkyl; and one of R⁷ and R⁸ is hydrogen and the other of R⁷ and R⁸ is C₁₋₆alkyl.

17. The method of claim 13, wherein R⁶ is —OR¹⁰ and R¹⁰ is chosen from C₁₋₄ alkyl, cyclohexyl, and phenyl.

18. The method of claim 13, wherein R⁶ is chosen from methyl, ethyl, n-propyl, and isopropyl; and one of R⁷ and R⁸ is hydrogen and the other of R⁷ and R⁸ is chosen from methyl, ethyl, n-propyl, and isopropyl.

19. The method of claim 13, wherein the compound is chosen from:
ethoxycarbonyloxyethyl methyl (2E)but-2-ene-1,4-dioate;
methyl (methylethoxycarbonyloxy)ethyl (2E)but-2-ene-1,4-dioate;
methyl (2-methylpropanoyloxy)ethyl (2E)but-2-ene-1,4-dioate;
methyl phenylcarbonyloxyethyl (2E)but-2-ene-1,4-dioate;
cyclohexylcarbonyloxybutyl methyl (2E)but-2-ene-1,4-dioate;
[(2E)-3-(methoxycarbonyl)prop-2-enoyloxy]ethyl methyl (2E)but-2-ene-1,4-dioate;
(cyclohexyloxycarbonyloxy)ethyl methyl (2E)but-2-ene-1,4-dioate;
methyl 2-methyl-1-phenylcarbonyloxypropyl (2E)but-2-ene-1,4-dioate;
3-({[(2E)-3-(methoxycarbonyl)prop-2-enoyloxy]methyl}oxycarbonyl)(3S)-3-aminopropanoic acid;
3-({[(2E)-3-(methoxycarbonyl)prop-2-enoyloxy]methyl}oxycarbonyl)(2S)-2-aminopropanoic acid;
3-({[(2E)-3-(methoxycarbonyl)prop-2-enoyloxy]methyl}oxycarbonyl)(3S)-3-(2-aminoacetylamino)propanoic acid;
3-({[(2E)-3-(methoxycarbonyl)prop-2-enoyloxy]methyl}oxycarbonyl)(2S)-2-aminopropanoic acid;
3-{[(2E)-3-(methoxycarbonyl)prop-2enoyloxy]ethoxycarbonyloxy}(2S)-2-aminopropanoic acid; and
a pharmaceutically acceptable salt of any of the foregoing.

20. The method of claim 3, wherein the compound is a compound of Formula (V): 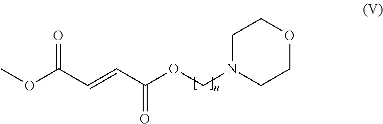
or a pharmaceutically acceptable salt thereof, wherein n is an integer from 2 to 6.

21. The method of claim 20, wherein n is chosen from 2 and 3.

22. The method of claim 20, wherein the compound is chosen from:
methyl (morpholinoethyl)fumarate;
methyl (morpholinopropyl)fumarate;
methyl (morpholinobutyl)fumarate; and
a pharmaceutically acceptable salt of any of the foregoing.

23. The method of claim 1, wherein the tumor is a solid tumor.

24. The method of claim 23, wherein the solid tumor is one of mamma carcinoma, colon carcinoma, melanoma, primary liver cell carcinoma, adenocarcinoma, kaposi's sarcoma, prostate carcinoma, multiple myeloma (plasmocytoma), Burkitt lymphoma, and Castleman tumor.

25. A method of treating a disease selected from cutaneous lupus erythematosus, lichen planus, macular degeneration, necrobiosis lipoidosis and neuromyelitis optica in a patient, comprising administering to a patient in need of such treatment a therapeutically effective amount of a compound selected from monomethyl fumarate and a monomethyl fumarate prodrug of Formulae (I), (II) or (V): 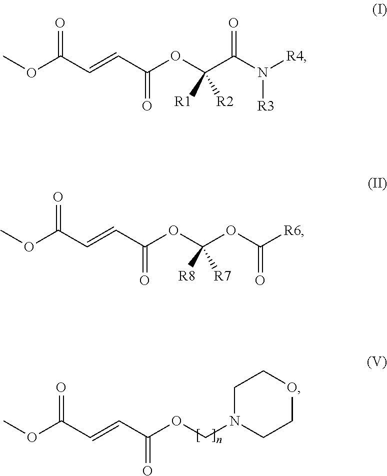
or a pharmaceutically acceptable salt thereof, wherein:
R¹ and R² are independently chosen from hydrogen, C₁₋₆ alkyl, and substituted C₁₋₆ alkyl;
R³ and R⁴ are independently chosen from hydrogen, C₁₋₆ alkyl, substituted C₁₋₆ alkyl, C₁₋₆ heteroalkyl, substituted C₁₋₆ heteroalkyl, C₄₋₁₂ cycloalkylalkyl, substituted C₄₋₁₂ cycloalkylalkyl, C₇₋₁₂ arylalkyl, and substituted C₇₋₁₂ arylalkyl; or R³ and R⁴ together with the nitrogen to which they are bonded form a ring chosen from a C₅₋₁₀ heteroaryl, substituted C₅₋₁₀ heteroaryl, C₅₋₁₀ heterocycloalkyl, and substituted C₅₋₁₀ heterocycloalkyl;
R⁶ is chosen from C₁₋₆alkyl, substituted C₁₋₆alkyl, C₁₋₆heteroalkyl, substituted C₁₋₆ heteroalkyl, C₃₋₈cycloalkyl, substituted C₃₋₈cycloalkyl, C₆₋₈aryl, substituted C₆₋₈aryl, and —OR¹⁰ wherein R¹⁰ is chosen from C₁₋₆alkyl, substituted C₁₋₆alkyl, C₃₋₁₀ cycloalkyl, substituted C₃₋₁₀ cycloalkyl, C₆₋₁₀ aryl, and substituted C₆₋₁₀ aryl;
R⁷ and R⁸ are independently chosen from hydrogen, C₁₋₆alkyl, and substituted C₁₋₆ alkyl;
n is an integer from 2 to 6; and
wherein each substituent group is independently chosen from halogen, —OH, —CN, —CF₃, ═O, —NO₂, benzyl, —C(O)NR¹¹₂, —R¹¹, —OR¹¹, —C(O)R¹¹, —COOR¹¹, and —NR¹¹₂ wherein each R¹¹ is independently chosen from hydrogen and C₁₋₄ alkyl.

26. A method of treating a disease selected from adrenal leukodystrophy, Alexanders Disease, Alpers' Disease, Canavan disease, chronic inflammatory demyelinating polyneuropathy, chronic lymphocytic leukemia, globoid cell leukodystrophy, hepatitis C viral infection, herpes simplex viral infection, human immunodeficiency viral infection, optic neuritis, Pelizaeus-Merzbacher disease, primary lateral sclerosis, progressive supranuclear palsy, Schilder's Disease, a tumor and Zellweger syndrome, comprising administering to a patient in need of such treatment a therapeutically effective amount of a compound selected from a monomethyl fumarate prodrug of Formulae (I), (II) or (V): 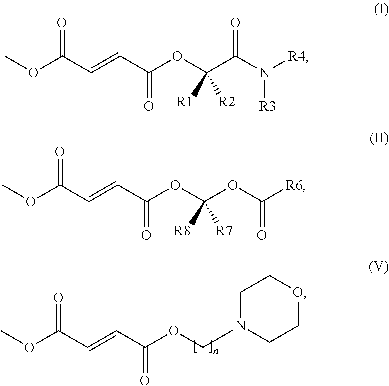
or a pharmaceutically acceptable salt thereof, wherein:
R¹ and R² are independently chosen from hydrogen, C₁₋₆ alkyl, and substituted C₁₋₆ alkyl;
R³ and R⁴ are independently chosen from hydrogen, C₁₋₆ alkyl, substituted C₁₋₆ alkyl, C₁₋₆ heteroalkyl, substituted C₁₋₆ heteroalkyl, C₄₋₁₂ cycloalkylalkyl, substituted C₄₋₁₂ cycloalkylalkyl, C₇₋₁₂ arylalkyl, and substituted C₇₋₁₂ arylalkyl; or R³ and R⁴ together with the nitrogen to which they are bonded form a ring chosen from a C₅₋₁₀ heteroaryl, substituted C₅₋₁₀ heteroaryl, C₅₋₁₀ heterocycloalkyl, and substituted C₅₋₁₀ heterocycloalkyl;
R⁶ is chosen from C₁₋₆ alkyl, substituted C₁₋₆ alkyl, C₁₋₆ heteroalkyl, substituted C₁₋₆ heteroalkyl, C₃₋₈cycloalkyl, substituted C₃₋₈cycloalkyl, C₆₋₈aryl, substituted C₆₋₈aryl, and —OR¹⁰ wherein R¹⁰ is chosen from C₁₋₆ alkyl, substituted C₁₋₆ alkyl, C₃₋₁₀ cycloalkyl, substituted C₃₋₁₀ cycloalkyl, C₆₋₁₀ aryl, and substituted C₆₋₁₀ aryl;
R⁷ and R⁸ are independently chosen from hydrogen, C₁₋₆ alkyl, and substituted C₁₋₆ alkyl;
n is an integer from 2 to 6; and
wherein each substituent group is independently chosen from halogen, —OH, —CN, —CF₃, ═O, —NO₂, benzyl, —C(O)NR¹¹₂, —R¹¹, —OR¹¹, —C(O)R¹¹, —COOR¹¹, and —NR¹¹₂ wherein each R¹¹ is independently chosen from hydrogen and C₁₋₄ alkyl.
